(19) United States
(12) Patent Application Publication
(10) Pub. No.: US 2021/0371389 A1
(43) Pub. Date: Dec. 2, 2021

(54) ONE-STEP SYNTHESIS OF SOYBEAN POLYOLS

(71) Applicant: Kansas Soybean Commission, Topeka, KS (US)

(72) Inventor: Santimukul Santra, Pittsburg, KS (US)

(21) Appl. No.: 17/238,951

(22) Filed: Apr. 23, 2021

Related U.S. Application Data
(60) Provisional application No. 63/015,167, filed on Apr. 24, 2020.

(51) Int. Cl. C07D 249/04; C11C 3/00

(52) U.S. Cl. CPC C07D 249/04; C11C 3/006

(57) ABSTRACT

A method of producing a triazoline-containing compound, the method comprising reacting an alkene, which comprises at least one a C═C double bond, with an azido compound, which comprises an azide anion having the chemical formula N₃⁻, wherein the alkene and the azido compound are constituents of a reaction mixture, so that a C—C single bond forms between the carbon atoms of the at least one C═C double bond and each of carbon atom of the C—C single bond also has a single bond with a different nitrogen atom of the azide anion thereby producing the triazoline-containing compound.

### CROSS-REFERENCE TO RELATED APPLICATIONS

This application claims the benefit of U.S. Provisional Application Ser. No. 63/015,167 filed on Apr. 24, 2020, which is incorporated herein by reference in its entirety.

### FIELD OF INVENTION

The present invention is directed to the production of soybean polyols.

### BACKGROUND OF INVENTION

Soybean polyols are important starting materials (synthons) to many soy-based products (e.g., foams, sealants, paints, adhesives, elastomers, etc.). To date, soybean polyols are typically synthesized from soybean oil (SBO) using at least two or three organic reaction steps. Examples of know reaction categories for producing polyols include Epoxidation and Ring Opening (FIG. 44), Hydroformylation and Hydrogenation (FIG. 45), and Ozonolysis and Hydrogenation (FIG. 46). These known synthesis methods are expensive, require catalysts and solvents, and are labor intensive, which ultimately makes the SBO-based products more expensive than many alternative petroleum-based products.

Thus, a need exists for method of preparing soybean polyols in a more efficient, less costly manner.

### SUMMARY OF INVENTION

In one embodiment, the present invention is directed to a method of producing a triazoline-containing compound. Said triazoline-containing compound may be a polyol based on an alkene such as soybean oil. The method comprises reacting an alkene, which comprises at least one a C═C double bond, with an azido compound, which comprises an azide anion having the chemical formula N₃⁻, wherein the alkene and the azido compound are constituents of a reaction mixture, so that a C—C single bond forms between the carbon atoms of the at least one C═C double bond and each carbon atom of the C—C single bond also has a single bond with a different nitrogen atom of the azide anion thereby producing the triazoline-containing compound according to Scheme I,

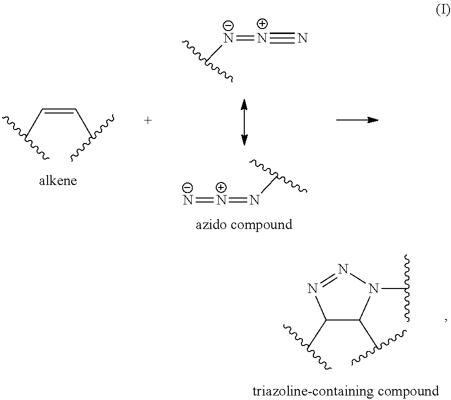

wherein the triazoline-containing compound is a constituent of a product mixture.

In an embodiment, the alkene is selected from the group consisting of triglycerides, small molecule aliphatic alkenes, terminal alkenes, and combinations thereof.

In an embodiment, the triglycerides are one or more vegetable oils selected from the group consisting of soybean oil, corn oil, palm oil, sunflower oil, canola oil, sesame oil, peanut oil, olive oil, cottonseed oil, avocado oil, almond oil, walnut oil, flaxseed oil, and combinations thereof.

In an embodiment, the azido compound further comprises a functional group selected from the group consisting of a hydroxyl group, an alkyl group, an amine group, a thiol group, and an ether group.

In an embodiment, the azido compound further comprises a hydroxyl functional group and is selected from the group consisting of propylene oxide azide, alkyl azides, alkane diazides, functional alkyl azides, and combinations thereof.

In an embodiment, the reaction mixture is free of a solvent.

In an embodiment, the reaction mixture is free of any other reagent.

In an embodiment, the reaction mixture is free of a catalyst.

In an embodiment, the reaction mixture is free of an initiator.

In an embodiment, the reaction mixture consists of the alkene and the azido compound.

In an embodiment, the step of reacting the alkene and the azido compound comprises adding an effective amount of energy to the reaction mixture to cause the reaction between the alkene and the azido compound for a desired duration.

In an embodiment, the effective amount of energy added to the reaction mixture by maintaining the reaction mixture at a temperature in a range of about 75° C. to about 180° C. and the desired duration is in a range from about 12 hours to about 48 hours.

In an embodiment, the effective amount of energy added to the reaction mixture by exposing the reaction mixture to ultraviolet light with a wavelength in a range of about 200 nm to about 400 nm.

In an embodiment, the effective amount of energy added to the reaction mixture by exposing the reaction mixture to microwave irradiation.

In an embodiment, the product mixture contains at least 90% by weight of the triazoline-containing compound.

In one embodiment, present invention is directed to a triazoline-containing triglyceride molecule comprising a glycerol-based backbone moiety and three fatty acid-based chain moieties bound to the glycerol-based backbone moiety via ester bonds, wherein at least one of the fatty acid-based chain moieties comprises at least one triazoline moiety that comprises a 5-membered heterocycle ring of two carbon atoms and three nitrogen atoms, wherein the two carbon atoms are also adjacent carbon atoms of the fatty acid-based chain moiety.

In one embodiment, the present invention is directed to a vegetable oil-based polyol molecule comprising:

a triglyceride moiety that comprises a glycerol-based backbone and three fatty acid-based chains bound to the glycerol-based backbone via ester bonds; and

at least one triazoline moiety that comprises:
a 5-membered heterocycle ring of two carbon atoms and three nitrogen atoms in which the two carbon atoms are also adjacent carbon atoms of one of the fatty acid-based chains of the triglyceride moiety; and
a hydroxyl functional group.

### BRIEF DESCRIPTION OF THE DRAWINGS

FIG. 1 is a FT-IR spectrum of 1-hexylazide showing the presence of an azide functional group.

FIG. 2 is a NMR spectrum of 1-Hexylazide showing the structure of an azide functional group.

FIG. 3 is gel permeation chromatography (GPC) results of the thermal stability of 1-hexylazide.

FIG. 4 is gel permeation chromatography (GPC) results of the thermal stability of 1-decene.

FIG. 5 is thin later chromatography (TLC) results of an unpurified 1-hexylazide+decene reaction product and GPC results of the unpurified 1-hexylazide+decene reaction product at 0 hour and 24 hours.

FIG. 6 is FT-IR spectra of a purified product of a reaction between decene and hexylazide.

FIG. 7 is a NMR spectrum of a product of a reaction between decene and hexylazide.

FIG. 8 is GPC results of a product of a reaction between +decene reaction product.

FIG. 9 is a FT-IR spectrum of a product of a reaction between decene and phenyl propyl azide.

FIG. 10 is a NMR spectrum of a product of a reaction between decene and phenyl propyl azide.

FIG. 11 is a GC-MS spectrum of a product of a reaction between decene and phenyl propyl azide.

FIG. 12 is GPC results of the starting materials of reaction between 4-phenyl-1-butene and hexyl azide.

FIG. 13 is GPC results of a reaction between 4-phenyl-1-butene and hexyl azide.

FIG. 14 is a NMR spectrum of a product of a reaction between 4-phenyl-1-butene and hexyl azide.

FIG. 15 is a FT-IR spectrum of a product of a reaction between 4-phenyl-1-butene and hexyl azide.

FIG. 16 is a FT-IR spectrum of propylene oxide azide.

FIG. 17 is a NMR spectrum of propylene oxide azide.

FIG. 18 is GPC results of a reaction between 4-phenyl-1-butene and propylene oxide azide.

FIG. 19 is time-dependent GPC results of a reaction between 4-phenyl-1-butene and propylene oxide azide.

FIG. 20 is a FT-IR spectrum of a reaction between 4-phenyl-1-butene and propylene oxide azide.

FIG. 21 is a NMR spectrum of 4-phenyl-1-butene.

FIG. 22 is NMR spectrum of a product of a reaction between 4-phenyl-1-butene and propylene oxide azide.

FIG. 23 is FT-IR spectrum of a soybean polyol take 24 hours after a reaction between a soybean oil and propylene oxide azide.

FIG. 24 is GPC results of soybean polyol synthesized according to one embodiment of the present invention at the indicated reaction times.

FIG. 25 is GPC results comparison of soybean polyol synthesized according to one embodiment of the present invention (black) and a commercial soybean polyol (red).

FIG. 26 is GPC results of a soybean polyol synthesized according to one embodiment of the present invention.

FIG. 27 is GPC results of a soybean polyol synthesized according to one embodiment of the present invention.

FIG. 28 is GPC results of a conventional soybean polyol.

FIG. 29 is GPC results of a soybean polyol synthesized according to one embodiment of the present invention.

FIG. 30 is GPC results of a soybean polyol synthesized according to one embodiment of the present invention and a conventional soybean polyol.

FIG. 31 is an image of rigid polyurethane foams based on soybean polyols.

FIG. 32 is an image of rigid polyurethane foams based on soybean polyols.

FIG. 33 is an image of rigid polyurethane foams based on soybean polyols.

FIG. 34 is an image of rigid polyurethane foams based on soybean polyols.

FIG. 35 is an image of rigid polyurethane foams before and after a burn test.

FIG. 36 is GPC results of a soybean polyol synthesized according to one embodiment of the present invention.

FIG. 37 is images of cast polyurethanes based on soybean polyols synthesized according to embodiment of the present invention.

FIG. 38 is a DSC diagram of a first cast polyurethane based on soybean polyol synthesized according to embodiment of the present invention.

FIG. 39 is a DSC diagram of a second cast polyurethane based on soybean polyol synthesized according to embodiment of the present invention.

FIG. 40 is a DSC diagram of a third cast polyurethane based on soybean polyol synthesized according to embodiment of the present invention.

FIG. 41 is a thermogravimetric analysis (TGA) diagram of a said first cast polyurethane.

FIG. 42 is a TGA diagram of said second cast polyurethane.

FIG. 43 is a TGA diagram of said third cast polyurethane.

FIG. 44 depicts the known Epoxidation and Ring Opening reactions for forming polyols.

FIG. 45 depicts the known Hydroformylation and Hydrogenation reactions for forming polyols.

FIG. 46 depicts the known Ozonolysis and Hydrogenation reactions for forming polyols.

FIG. 47 depicts two Click-ene reaction embodiments for forming polyols accord to the method of the present invention.

FIG. 48 is GPC results of a soybean polyol one-step synthesized using a 20 watt UV-LED light.

FIG. 49 is GPC results of a soybean polyol one-step synthesize using a 1000 watt microwave.

### DETAILED DESCRIPTION OF INVENTION

In one embodiment, the present invention is directed to a method of producing a triazoline-containing compound. Said triazoline-containing compound may be a polyol based on an alkene such as soybean oil. The method comprises reacting an alkene, which comprises at least one a C═C double bond, with an azido compound, which comprises an azide anion having the chemical formula N₃⁻, wherein the alkene and the azido compound are constituents of a reaction mixture, so that a C—C single bond forms between the carbon atoms of the at least one C═C double bond and each carbon atom of the C—C single bond also has a single bond with a different nitrogen atom of the azide anion thereby producing the triazoline-containing compound according to Scheme I,

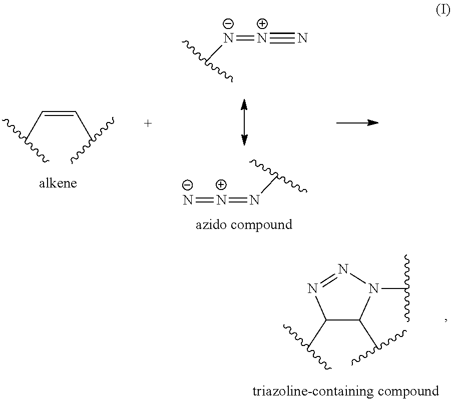

wherein the triazoline-containing compound is a constituent of a product mixture.

Advantageously, the above-described method provides one or more benefits compared to previously known methods of producing polyols. For example, previously known methods require at least two steps in the synthesis of the polyol whereas the synthesis of the present method may be conducted in a single step.

Additionally, the previously known methods require relatively expensive chemicals, solvents, and catalysts to produce the polyol whereas the method of the present invention may be conducted without solvents, expensive chemicals, and/or catalysts. In fact, in one embodiment, the reaction mixture is free of a solvent. In another embodiment, the reaction mixture is free of any other reagent. In yet another embodiment, the reaction mixture is free of a catalyst. In still another embodiment, the reaction mixture is free of an initiator. In another embodiment, the reaction mixture is free of a solvent, any other reagent, a catalyst, and an initiator. In yet another embodiment, the reaction mixture consists of the alkene and the azido compound.

Further, previously known methods of producing polyols required so-called “work-up” steps involving, for example, separation of solvent by extraction. In contrast, the method of the present invention may be conducted without such a work-up step.

Also, the previously known methods of producing polyols required purification of the reaction product before the polyols in the reaction product could be used in the production of other commercial products. In contrast, the although the reaction product of the present invention, which contains polyol molecules, may be purified, the reaction product can, depending upon the application, be used without additional purification.

The previously known methods of producing polyols also produced toxic byproducts such as nickel or platinum metals from the catalysts used. The method of the present invention may be performed without producing such toxic byproducts.

The previously known methods of producing polyols tend to have relatively low yields (e.g., from 50% to 78%) whereas the method of the present invention may achieve yields of 80% to 95% or higher.

The present invention may also be conducted in simpler, easier to manufacture and operate facilities compared to facilities based on conventional production methods.

Any one or a combination of the foregoing benefits may contribute to polyols produced according to the present invention being produced and sold at a cost that is less than that of soybean oil-based polyols.

### Reaction Mixture

As indicated above, the reaction mixture comprises an alkene and an azido compound. Typically, the reaction would be conducted with a reaction mixture in which the alkene and the azido compound are at a stoichiometric equivalent ration of about 1:1. Although, the reaction may be conducted when the reaction mixture comprises an excess of either of the alkene and azido reactants, no benefit is believed to be realized by doing so.

Further, as indicated above, the reaction may be conducted with a reaction mixture that comprises other constituents (e.g., solvent(s), other reagent(s), catalyst(s), and initiator(s)) but they are not required. In fact, there are advantages to the reaction mixture being free of one or more or even all of them.

In one embodiment, the reaction mixture comprises at least 50 wt % of the alkene and azido compound combined. In another embodiment, the combined amount of the alkene and the azido compound is at least 75 wt % of the reaction mixture. In yet another embodiment, the reaction mixture may consist only of the alkene and the azido compound.

### Alkene

In one embodiment, the alkene is selected from the group consisting of triglycerides, small molecule aliphatic alkenes, terminal alkenes, and combinations thereof.

In another embodiment, the alkene is selected from the group consisting of small molecule aliphatic alkenes, terminal alkenes, unsaturated vegetable oils, and combinations thereof.

Exemplary triglycerides include one or more vegetable oils selected from the group consisting of soybean oil, corn oil, palm oil, sunflower oil, canola oil, sesame oil, peanut oil, olive oil, cottonseed oil, avocado oil, almond oil, walnut oil, flaxseed oil, and combinations thereof. In one embodiment, the triglyceride is selected from group consisting of soybean oil, corn oil, and combinations thereof.

Exemplary small molecule aliphatic alkenes include decene, acyclic and cyclic alkene derivatives, and combinations thereof. In one embodiment, the small molecule aliphatic alkenes are selected from group consisting of aliphatic and aromatic moieties, and combinations thereof.

Exemplary terminal alkenes include decene, phenyl alkyl alkene, and combinations thereof. In one embodiment, the terminal alkenes are selected from group consisting of aliphatic and aromatic alkenes and combinations thereof.

In another embodiment, the alkene is selected from the group consisting of decene, phenyl propyl alkene, soybean oil, corn oil, and combinations thereof.

In one embodiment, the alkene consists of one or more triglycerides.

In another embodiment, the alkene consists of soybean oil.

### Azido Compound

As indicated above, the azido compound comprises an azide anion having the chemical formula N₃⁻. The azido anion or functionality may be part of a cyclic, acyclic, heterocyclic compounds or a combination thereof. Exemplary azido compounds include hexyl azide, phenyl propyl azide, and combinations thereof.

In one embodiment, the azido compound further comprises a functional group selected from the group consisting of a hydroxyl group, an alkyl group, an amine group, a thiol group, and an ether group. Exemplary azido compounds with such a functional group include propylene oxide azide, amino propyl azide, thio butyl azide, and combinations thereof.

In one embodiment, the azido compound further comprises a hydroxyl functional group and is selected from the group consisting of propylene oxide azide, alkyl azides, alkane diazides, functional alkyl azides, and combinations thereof.

Exemplary alkyl azides include butyl azide, hexyl azide, octyl azide, decyl azide, and combinations thereof.

Exemplary alkane diazides include butyl diazide, hexyl diazide, octyl diazide, decyl diazide, and combinations thereof.

Exemplary functional alkyl azides include propylene oxide azide, amino propyl azide thio butyl azide, and combinations.

In one embodiment, the azido compound is selected from the group consisting of hexyl azide, propylene oxide azide, and combinations thereof.

### Conducting the Reaction

The step of reacting the alkene and the azido compound comprises adding an effective amount of energy to the reaction mixture to cause the reaction between the alkene and the azido compound for a desired duration. For example, in one embodiment, this is accomplished by maintaining the reaction mixture at a temperature in a range of about 75° C. to about 180° C. for a duration and the desired duration is in a range from about 12 hours to about 48 hours.

In another embodiment, the effective amount of energy added to the reaction mixture may be accomplished by exposing the reaction mixture to ultraviolet light with a wavelength in a range of about 200 nm to about 400 nm (between about 20 W and about 200 W) per 10 grams to 1,000 grams of reaction mixture for a duration in a range of about 12 hours to about 72 hours.

In another embodiment, wherein the effective amount of energy added to the reaction mixture by exposing the reaction mixture to microwave irradiation with a wavelength in a range of about 1×10⁶ nm to about 1×10⁸ nm at a power in a range of about 700 wats to about 1,200 watts per 1 gram to 100 grams of reaction mixture for a duration of up to about 3 hours, and preferably in a range of about 5 minutes to about 30 minutes.

In another embodiment, one may use a manner of driving the reaction selected from the group consisting of temperature, UV radiation, microwave radiation, and combinations thereof.

### Product Mixture

In one embodiment, upon completion of the reaction, the product mixture contains at least 90% by weight of the triazoline-containing compound.

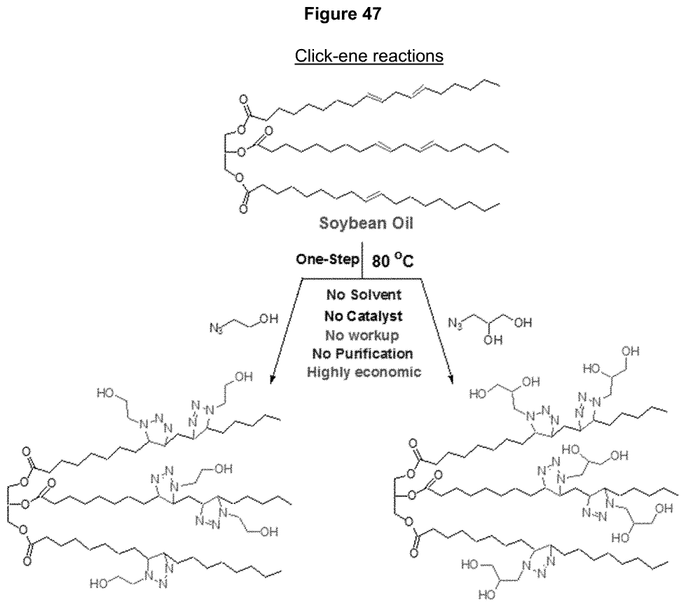

It is believed that the resulting triazoline-containing compound may have a novel structure. For example, if the alkene is a triglyceride, the resulting triazoline-containing compound is a triglyceride in which at least one of the C═C double bonds of the alkene is transformed to a C—C single bond and each carbon atom of the C—C single bond also has a single bond with a different nitrogen atom of the azide anion originally from the azido compound reactant. This product is depicted generally at the product of the Scheme I reaction set forth above. Additionally, this product is depicted more specifically in FIG. 47, which shows the reactions in which one azido compound comprises one hydroxyl group and another azido compound further comprises two hydroxyl groups.

In one embodiment, the above-described method is used to produce a triazoline-containing triglyceride molecule comprising a glycerol-based backbone moiety and three fatty acid-based chain moieties bound to the glycerol-based backbone moiety via ester bonds, wherein at least one of the fatty acid-based chain moieties comprises at least one triazoline moiety that comprises a 5-membered heterocycle ring of two carbon atoms and three nitrogen atoms, wherein the two carbon atoms are also adjacent carbon atoms of the fatty acid-based chain moiety.

As indicated above, in one embodiment, the at least one triazoline moiety further comprises a functional group selected from the group consisting of a hydroxyl group, an alkyl group, an amine group, a thiol group, and an ether group. In another embodiment, the at least one triazoline moiety comprises a functional group that is a hydroxyl group.

In one embodiment, two of the three fatty acid-based chain moieties comprise at least one triazoline moiety. In another embodiment, each of the three fatty acid-based chain moieties comprises at least one triazoline moiety.

In one embodiment, the at least one triazoline moiety further comprises a linking moiety between the 5-membered heterocycle ring and the functional group, wherein the linking moiety is selected from the group consisting of alkyl and aryl azide derivatives.

In one embodiment, the above-described method is used to produce a vegetable oil-based polyol molecule comprising:

a triglyceride moiety that comprises a glycerol-based backbone and three fatty acid-based chains bound to the glycerol-based backbone via ester bonds; and

at least one triazoline moiety that comprises:
a 5-membered heterocycle ring of two carbon atoms and three nitrogen atoms in which the two carbon atoms are also adjacent carbon atoms of one of the fatty acid-based chains of the triglyceride moiety; and
a hydroxyl functional group.

The vegetable oil may be selected from the group consisting of soybean oil, corn oil, palm oil, sunflower oil, canola oil, sesame oil, peanut oil, olive oil, cottonseed oil, avocado oil, almond oil, walnut oil, flaxseed oil, and combinations thereof. In one embodiment, the vegetable oil is soybean oil.

In one embodiment, the vegetable oil-based polyol molecule comprises at least one triazoline moiety with each fatty acid-based chain of the triglyceride portion.

In one embodiment, the at least one triazoline moiety further comprises a linking moiety between the 5-membered heterocycle ring and the functional group, wherein the linking moiety is selected from the group consisting of alkyl and aryl azide derivatives.

### EXAMPLES

### Example 1

This example is directed to reacting alkene and azide without using any solvent and catalyst. Towards this end, we selected the two simplest relevant molecules, hexyl azide and decene (Scheme 1) to validate our hypothesis and feasibility of the so-called “Click-ene” reaction disclosed herein.

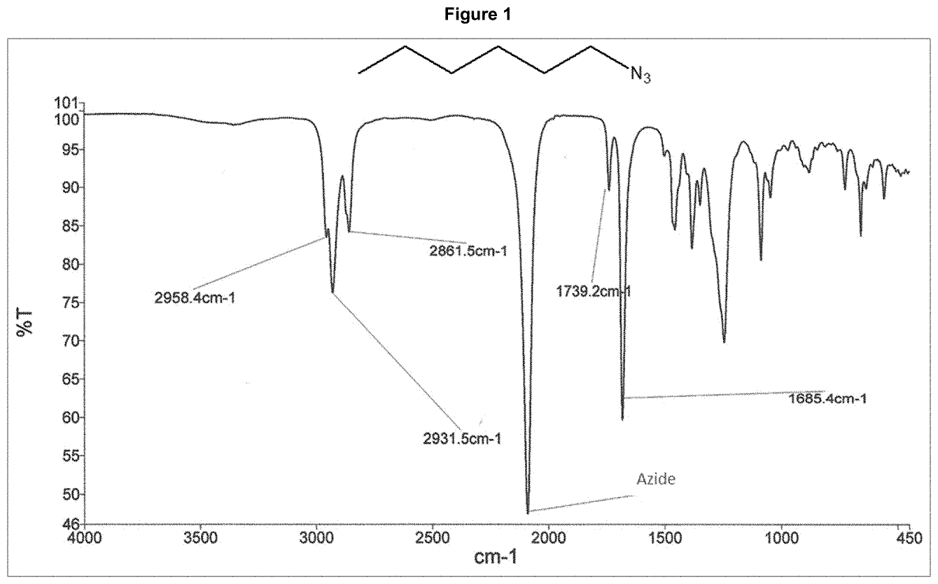

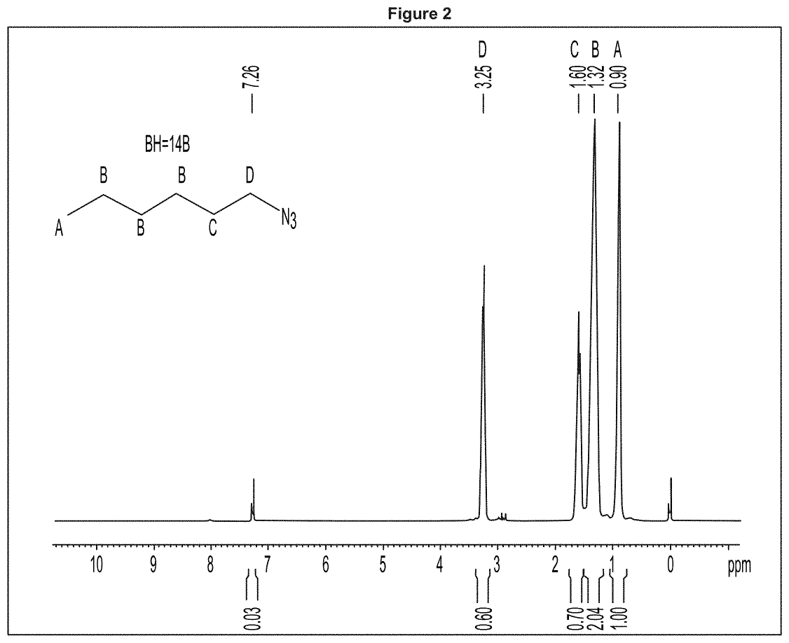

The hexyl azide was synthesized (Scheme 2) from hexyl bromide. The synthesized azide was characterized using Fourier transform infrared spectroscopy (FTIR) (FIG. 1) and nuclear magnetic resonance (NMR) (FIG. 2). The FT-IR spectrum confirmed the presence of the azide group and the NMR showed the structure as well as the purity.

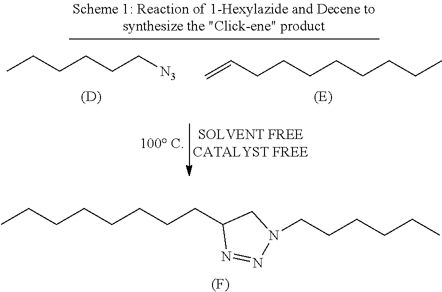

Synthesis of 1-hexylazide (C): 1-Bromohexane (A) (10.0 g, 0.060 mol) and sodium azide (B) (19.71 g, 0.303 mol) were added into a 250 mL round-bottom flask containing 60 mL of DMF. The reaction mixture was heated to 90 degrees Celsius for 48 hours with stirring. Upon completion of the 48 hours, the reaction mixture was brought to room temperature, the poured in water and extracted with ethyl acetate. The organic layer was washed with water, dried over Na₂SO₄, and concentrated in order to obtain the product.

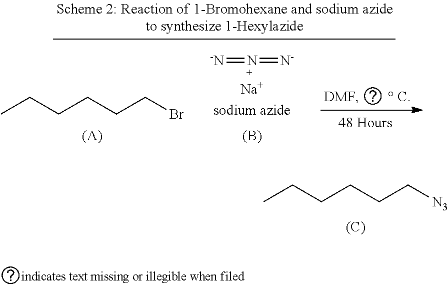

The 1-hexylazide was then characterized using FT-IR (FIG. 1) which confirmed the presence of the azide functional group (2099 cm⁻¹, as well as by NMR which confirmed the formation of the 1-hexylazide (FIG. 2).

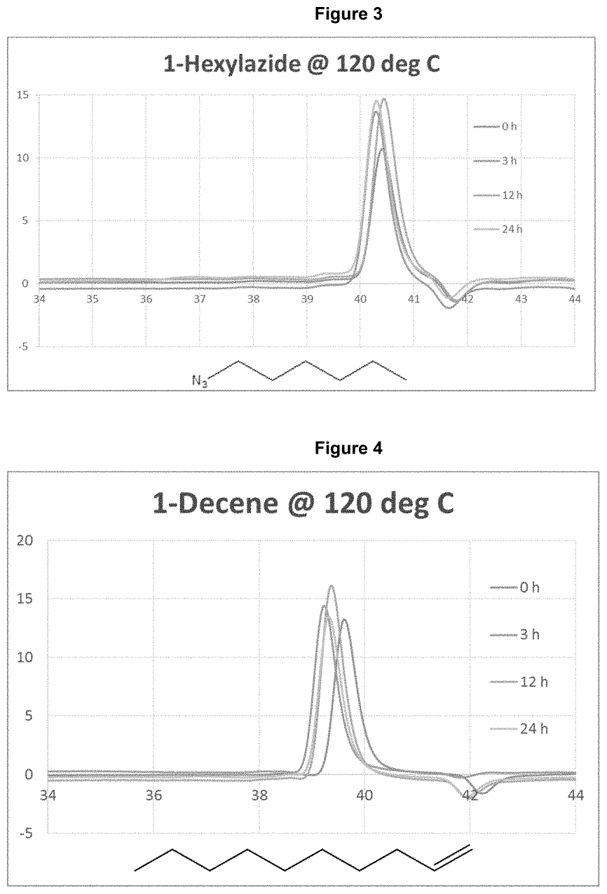

The solvent- and catalyst-free “Click-ene” chemistry/reaction may be conducted at an elevated temperature. But it was desirable to determine the stability of the reactants (i.e., hexyl azide and decene) at the elevated temperature. The reactions were conducted by heating the reactants in a flask at more than 100° C. (e.g., at 120° C.) and the change in molecular weight, if any, was monitored by gel permeation chromatography (GPC), with a constant time interval. The GPC chromatograms showed that hexyl azide (FIG. 3) and 1-decene (FIG. 4) were stable at that elevated temperature, and no degraded or polymeric products were obtained by the thermal stability experiments. This suggests that the selected small molecules are stable above 100° C. and therefore, we can perform “Click-ene” chemistry at that temperature without any potential degradation or decomposition of the starting materials.

Synthesis of Click-ene Product: The reaction between the azide compound and alkene compound (i.e. the so-called “Click-ene” chemistry), was performed at 100° C. without using any solvent and catalyst. Briefly, 1-hexylazide (D) (2.0 g, 0.0158 mol) and decene (E) (2.20 g, 0.0158 mol) were added to a 50-mL round-bottom flask (Scheme 1). The reaction mixture was heated to 100° C. for 24 hours with stirring. Upon completion of the 24 hours, the reaction was cooled to room temperature and the stirring was stopped. Advantageously, a laborious work-up step was not necessary as the reaction was conducted without any solvent and catalysts. Similar results were obtained when the reaction carried out at 80° C.

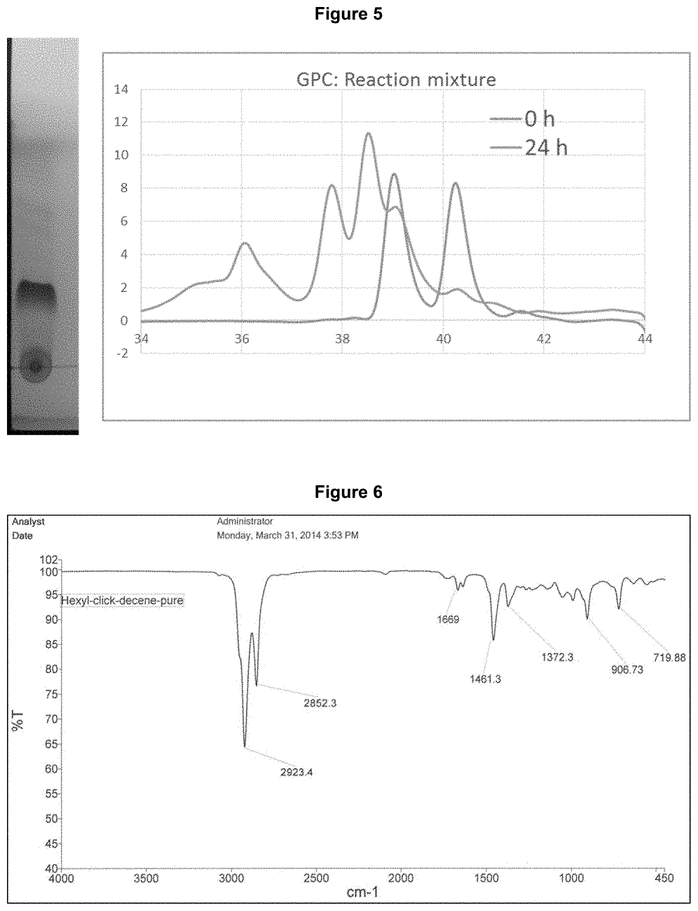

After the reaction, thin layer chromatography (TLC) was performed on the final product, and compared with the starting materials. The TLC showed the formation of a new dark spot (TLC, left, FIG. 5). It also showed very minimal amounts of the starting materials, indicating the reaction was not 100% complete. However, it was desirable to see a new dark spot on the TLC, which might have been because the product had a triazole ring (F, Scheme 1).

For further confirmation of the formation of new product(s), GPC experiments with the reaction were carried out. In a typical GPC experiment, sample was injected at the beginning of the reaction (0 hours) and at the end of the reaction (24 hours). The GPC results after 24 hours showed the formation of new bands in the GPC chromatogram (FIG. 5), which indicated the formation of new compounds that could have been the projected “Click-ene” product. To further confirm that the reaction proceeded as desired, the pure sample was characterized by FT-IR (FIG. 6). The result showed that the azide band at 2099 cm⁻¹ for the hexyl azide starting material had been greatly reduced in strength when compared to the original FT-IR spectrum of the 1-hexylazide (FIG. 1), which showed the progress of the reaction between the double bond and the azide function group.

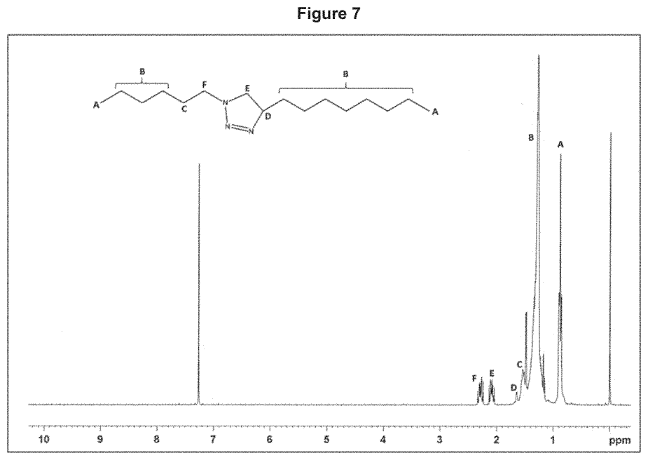

Characterization of the “Click-ene” product by NMR spectroscopy: The reaction product was more than 95% pure. To acquire pure NMR spectrum, flash column chromatography was used to further purify the product. The “Click-ene” product was dried under vacuum for 6 hours in order to remove any solvent and other volatile chemicals. The sample (10 mg) was dissolved in CDCl₃ and 300 MHz NMR from Bruker was used for obtaining the spectrum as shown in FIG. 7.

The results show a successful reaction condition for the “click-ene” chemistry in which more than 95% pure product was produced in one-step without using any solvent or catalyst.

### Example 2

In this example, phenyl propyl azide was used as an alternative to hexyl azide.

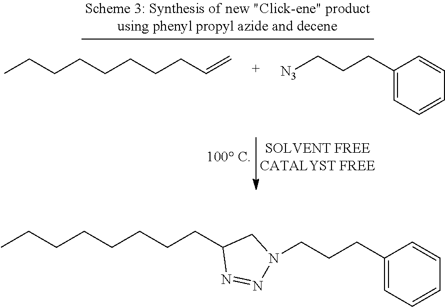

Procedure: The reaction was performed at 100° C. and without using any solvent and catalyst. Briefly, phenyl propyl azide (1 mol) and decene (1 mol) were added to a 50-mL round-bottom flask (Scheme 3). The reaction mixture was heated to 100° C. for 24 hours with stirring. Upon completion of the 24 hours, the reaction was cooled to room temperature and the stirring was stopped.

After the reaction, thin layer chromatography (TLC) was performed on the final product, and compared with the starting materials. The TLC showed the formation of a new dark spot (TLC). For further confirmation of the formation of new product(s), GPC experiments with the reaction were carried out. In a typical GPC experiment, sample was injected at the beginning of the reaction (0 hours) and at the end of the reaction (24 hours).

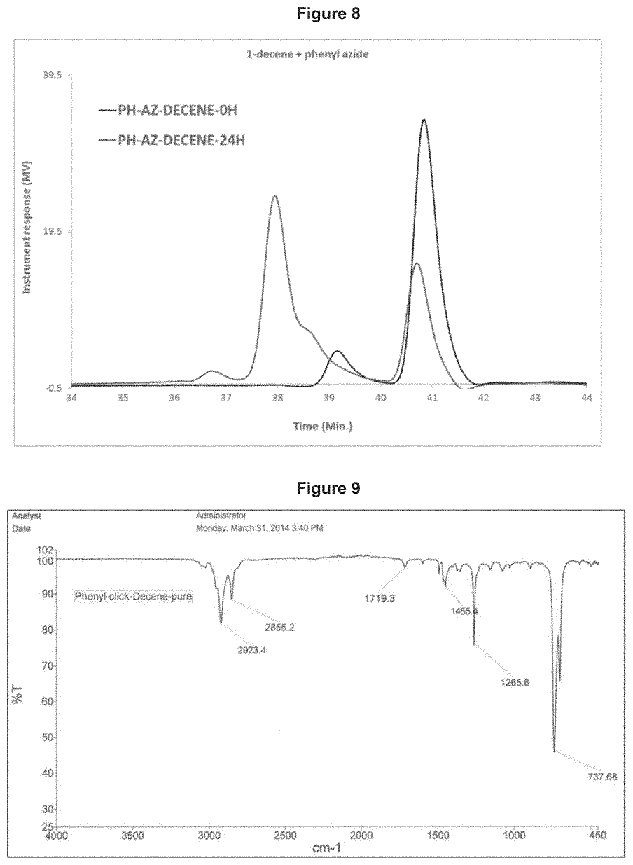

The GPC experiment confirmed that the formation of new product (red line, FIG. 8).

The FT-IR spectrum confirmed for progress of the reaction as after the reaction, azide band at 2100 cm⁻¹ was gone (FIG. 9).

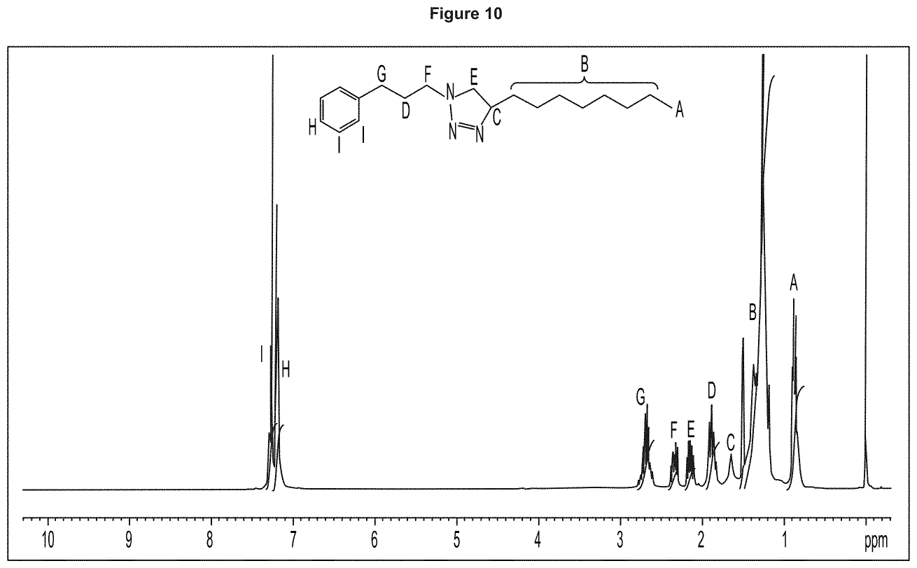

The reaction product was more than 96% pure, however, in order to acquire pure NMR spectrum, flash column chromatography was used to further purify the product. The “Click-ene” product was dried under vacuum for 6 hours in order to remove any solvent and other volatile chemicals. The sample (10 mg) was dissolved in CDCl₃ and 300 MHz NMR from Bruker was used for obtaining the spectrum as shown in FIG. 10. The NMR spectrum confirmed for the synthesis of proposed “Click-ene” product. Similar results obtained when the reaction was carried out at 80° C.

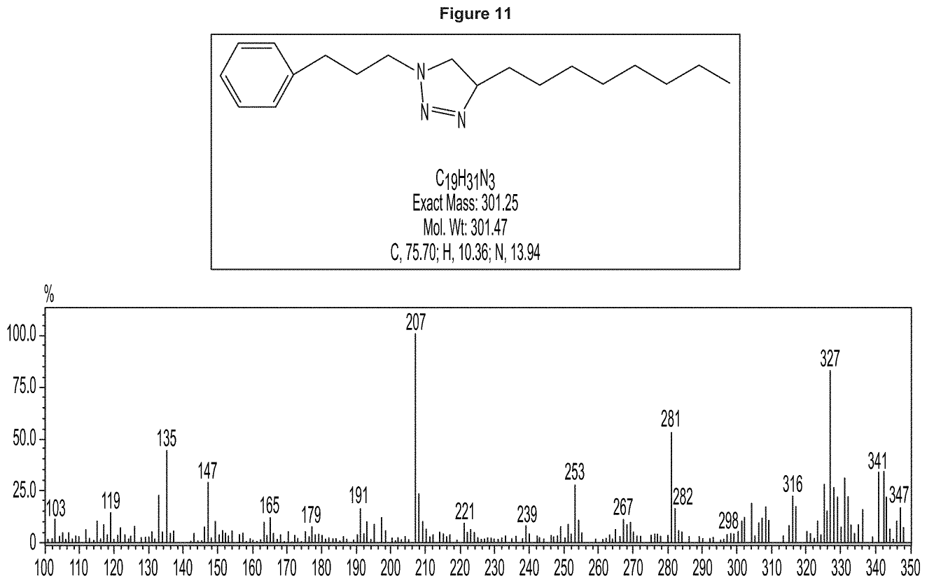

The product was also characterized by GC-MS experiment, showing the mass peak at 327 (M 301+Na 23+3H, FIG. 11), which further confirmed the formation of the desired product.

### Example 3

In this example, 4-phenyl-1-butene was used instead of the previously used decene.

Procedure: The reaction was performed at 100° C. and without using any solvent and catalyst. Briefly, hexyl azide (1 mol) and 4-phenyl-1-butene (1 mol) were added to a 50-mL round-bottom flask (Scheme 4). The reaction mixture was heated to 100° C. for 24 hours while being stirred. Upon completion of the 24 hours, the reaction was cooled to room temperature and the stirring was stopped.

After the reaction, thin layer chromatography (TLC) was performed on the final product, and compared with the starting materials. The TLC showed the formation of a new dark spot (TLC).

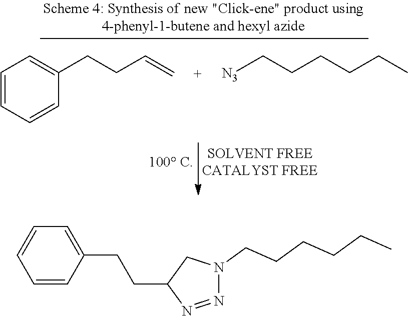

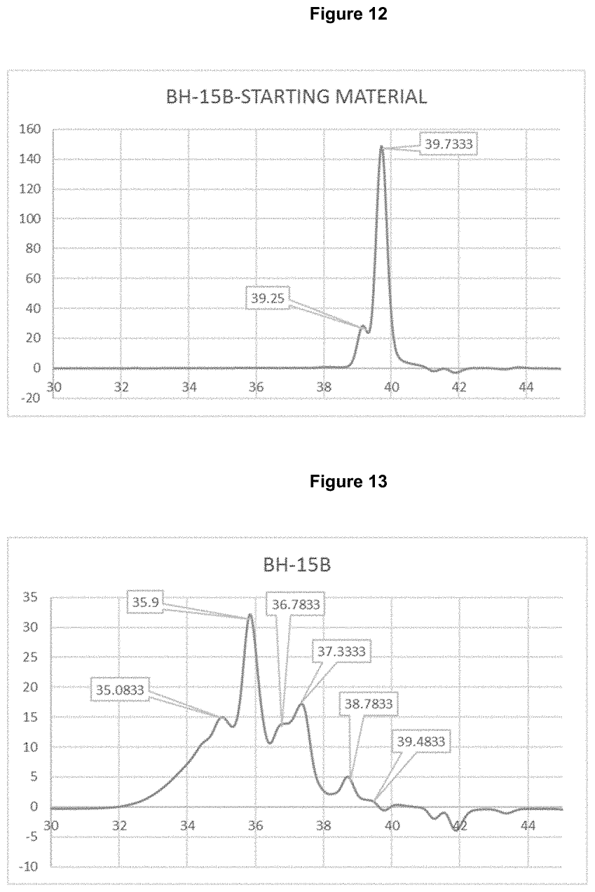

GPC experiments with the reaction were carried out to see the progress of the reaction. In a typical GPC experiment, sample was injected at the beginning of the reaction (0 hours, FIG. 12) showing the presence of starting materials only. After 24 hours of the reaction, GPC experiments were performed in order to see for the formation of any new products (FIG. 13). Results showed the formation of new peaks, potentially for the formation of the expected product.

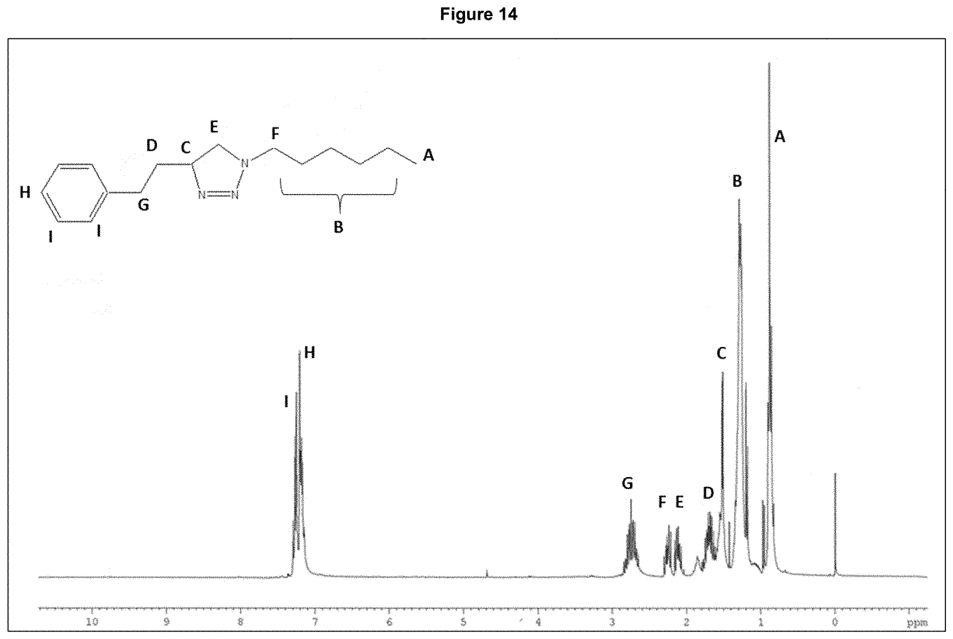

Characterization of the “Click-ene” product by NMR spectroscopy: The reaction product was more than 96% pure. Flash column chromatography was conducted to further purify the product. The “Click-ene” product was dried under vacuum for 6 hours in order to remove any solvent and other volatile chemicals. The sample (10 mg) was dissolved in CDCl₃ and 300 MHz NMR from Bruker was used for obtaining the spectrum as shown in FIG. 14.

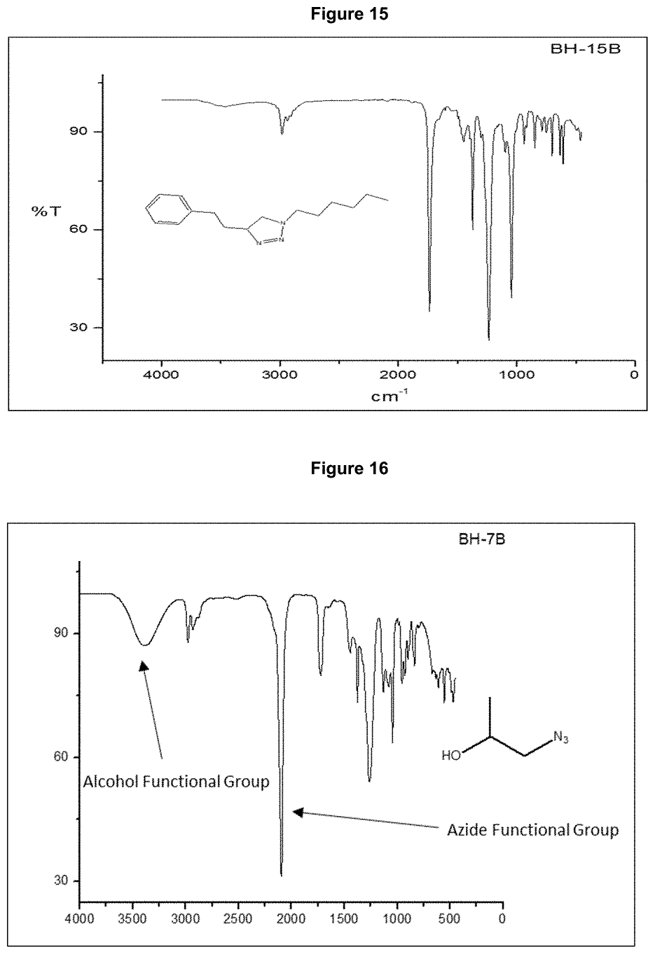

The FT-IR spectrum of FIG. 14 shows the absence of any azide band at around 2100 cm⁻¹, indicating the complete conversion of the azide reactant into product (FIG. 15). Similar results obtained when the reaction was carried out at 80° C.

### Example 4

In this example, 4-phenyl-1-butene and propylene oxide azide (PO-azide) were used. It is to noted that the PO-azide has terminal hydroxyl group (—OH group), which would result in the direct production of soybean polyols when reacted with soybean oil (SBO). This reaction was conducted to also determine whether the hydroxyl groups would interfere with the azide or the double bond at an elevated temperature.

Synthesis of Propylene Oxide Azide from Propylene Oxide (PO):

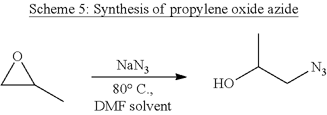

Procedure: The reaction was performed at 80° C. and using DMF solvent. Briefly, propylene oxide (1 mol) was added to DMF (50 mL) followed by addition of sodium azide (3 mol). This reaction mixture was heated at 80° C. and continued for 12 hours and the product was extracted using ethyl acetate solvent. The yield of this reaction was found to be 90%.

Characterization of the “propylene oxide azide” product by FT-IR spectroscopy: The FT-IR spectrum of FIG. 16 shows the presence of an azide band at around 2100 cm⁻¹ and a band at 3350 cm⁻¹, indicating the successful synthesis of propylene oxide.

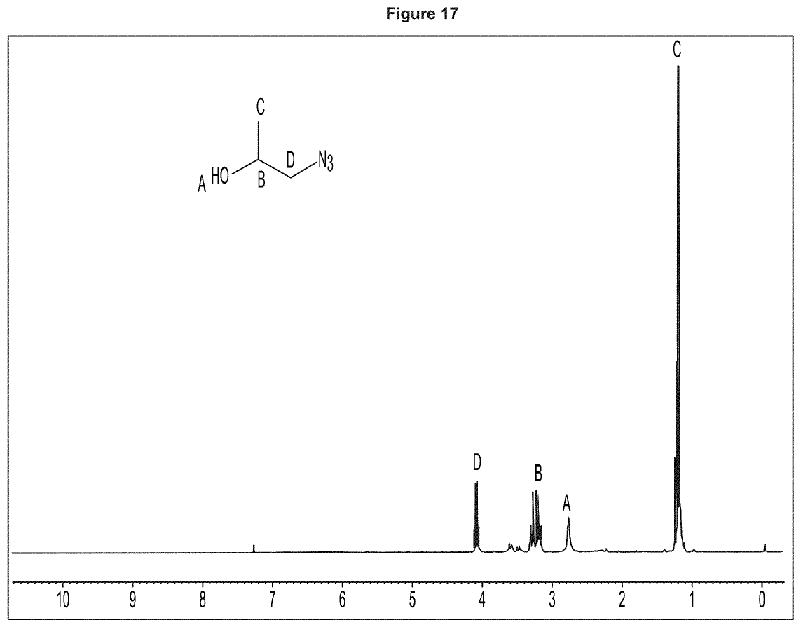

Characterization of the “propylene oxide azide” product synthesized in Scheme 5 using NMR spectroscopy: The successful formation of propylene oxide azide is further characterized by NMR spectroscopy as shown in FIG. 17.

Synthesis of “click-ene” product using 4-phenyl-1-butene and propylene oxide azide (PO-azide): As described earlier, the reaction between PO-azide and 4-phenyl-1-butene was performed without using catalyst and solvent (Scheme 6). The reaction mixture was continued at 100° C. for 24 hours. If successful, the resulting product would have a hydroxyl group.

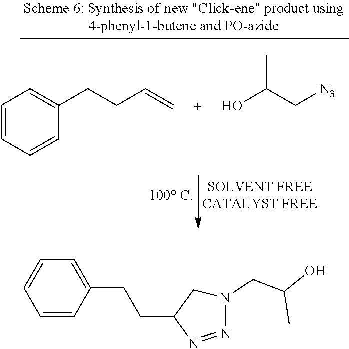

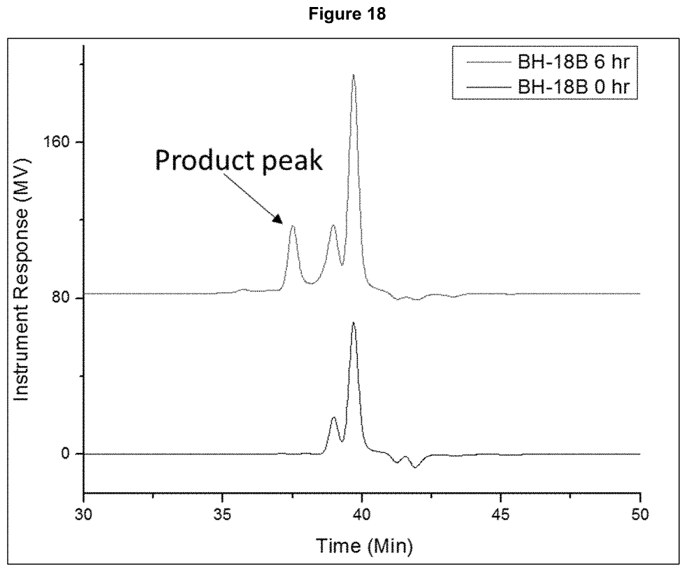

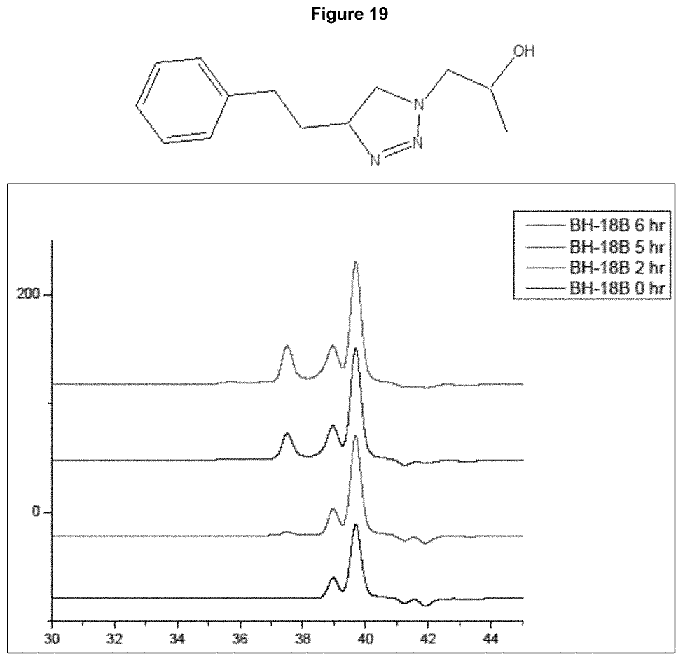

GPC experiments were carried out to see the progress of the reaction. In a typical GPC experiment, sample was injected at the beginning of the reaction (0 hours) showing the presence of starting materials only. After 6 hours of the reaction, GPC experiments were performed in order to see for the formation of any new products (FIG. 18 and FIG. 19). The results showed the formation of new peaks, potentially for the formation of the expected product.

The functional “click-ene” product was purified quickly using flash column chromatographic technique. The purified product was then subjected to a high vacuum overnight. The resulting pure product was characterized using various spectroscopic techniques.

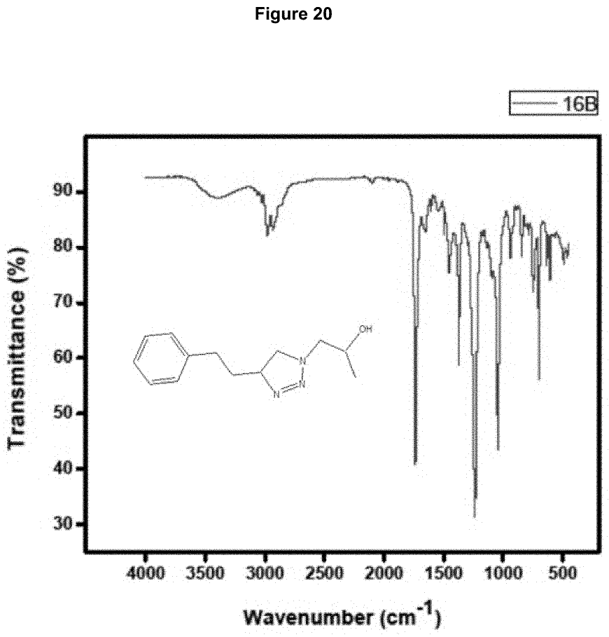

The FT-IR experiment resulted in a spectra demonstrating the successful synthesis of the targeted functional “click-ene” product. The presence of a hydroxyl stretching band at 3400 cm⁻¹, confirmed for the formation of the desired product (FIG. 20).

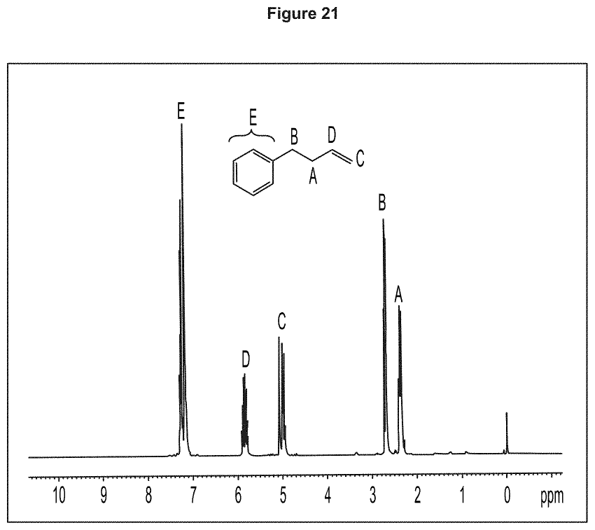

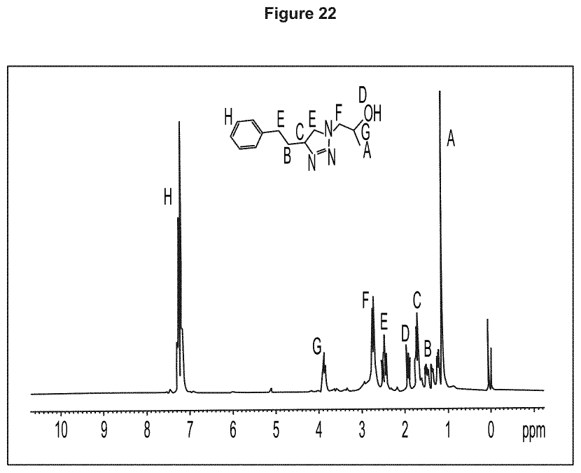

The hydroxyl functionalized “click-ene” product was further characterized by NMR spectroscopic method. FIG. 21, the NMR spectrum showed the purity of the starting material. The NMR spectra of the pure product was recorded and presented in FIG. 22. This NMR data further confirmed the successful synthesis of desired hydroxylated “click-ene” product.

### Example 5

This example is a one-step synthesis of soybean polyols from propylene oxide azide and soybean oil using the “Click-ene” chemistry without using any solvent or catalyst at 80° C. according to Scheme 7.

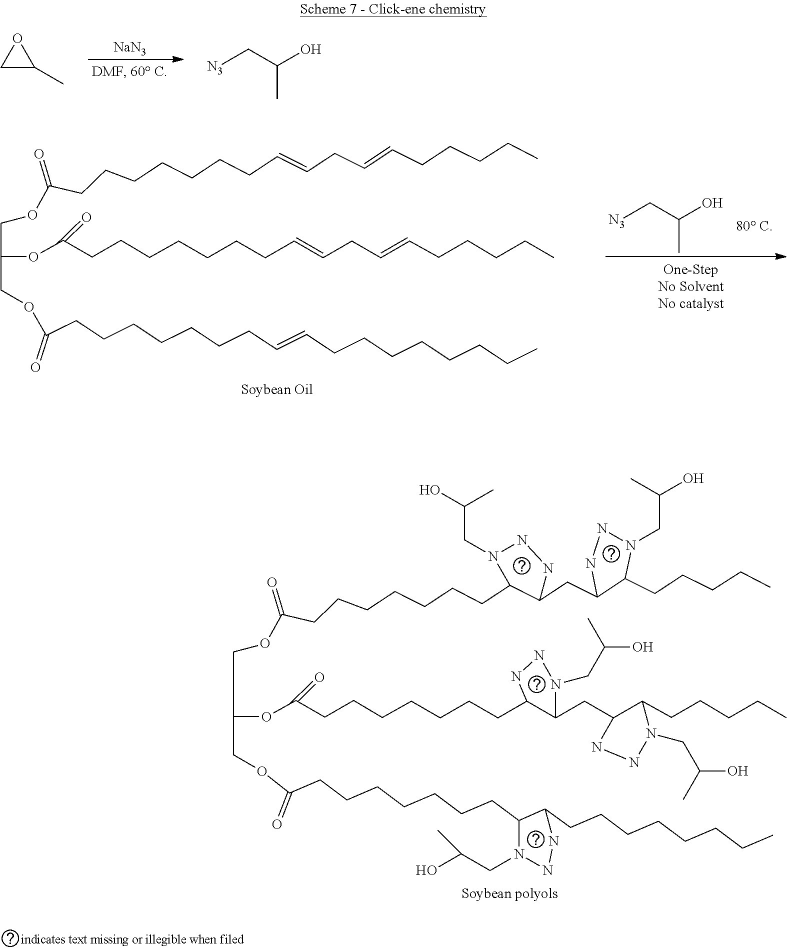

Procedure: The reaction between propylene oxide azide and soybean oil was performed at 80° C. and without using any solvent and catalyst. Briefly, propylene oxide azide (4.8 mol) and soybean oil (1 mol) were added to a 50-mL round-bottom flask (Scheme 7). The reaction mixture was heated to 80° C. for 24 hours with stirring. Upon completion of the 24 hours, the reaction was cooled to room temperature and the stirring was stopped. Samples were collected at different time intervals and characterized using various spectroscopic methods to determine the progress of the reaction.

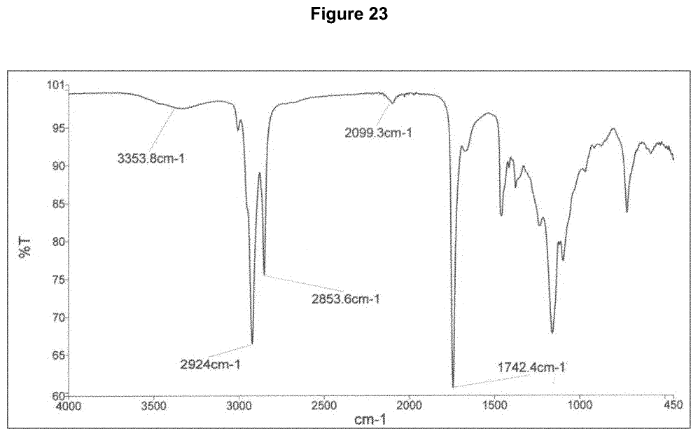

First, soybean polyols sample after 24 hours of reaction was analyzed using FT-IR, as shown in FIG. 23. The band for propylene oxide azide disappeared, which indicated that the reaction proceeded. In addition, the appearance of a band at 3353 cm⁻¹ confirmed the synthesis of soybean polyols.

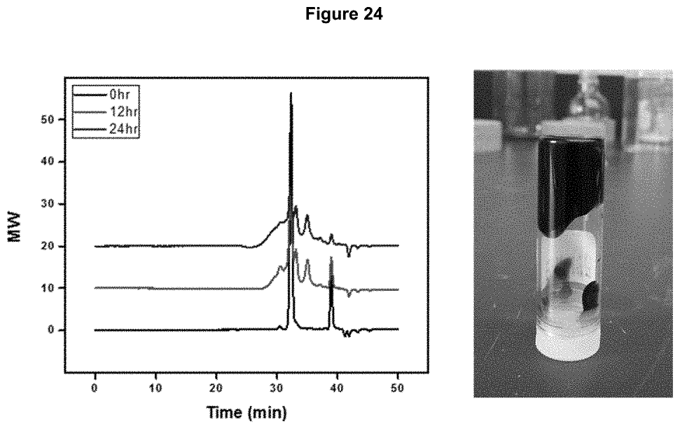

Next, the synthesized soybean polyols at different time points were characterized by gel permeation chromatography, as shown in FIG. 24. The appearance of new band over time shows the formation of a new product (i.e., the expected soybean polyols). After 24 hours of reaction, no substantial change in the GPC chromatograms were observed, which indicates that the reaction was completed within 24 hours and the product is stable at the reaction temperature.

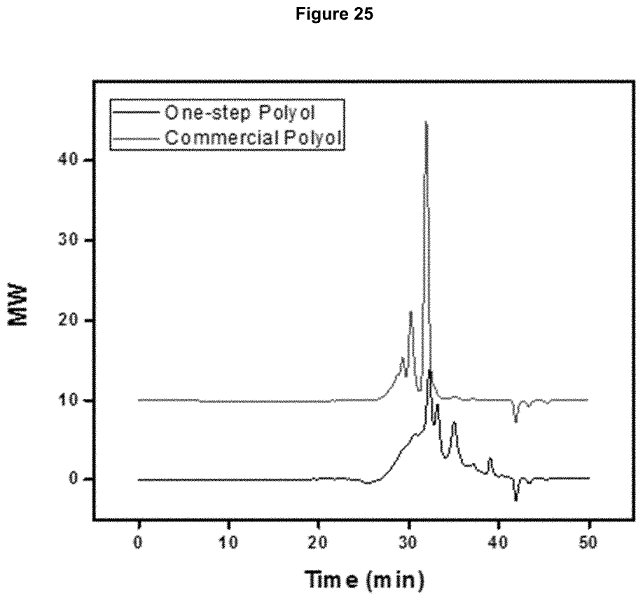

To confirm the obtained product is indeed the expected soybean polyols, the GPC chromatograms of obtained soybean polyols were compared to that of commercial polyols from Cargill, as presented in FIG. 25. Advantageously, the one-step soybean polyols prepared as described herein (i.e., black curve) yielded better than that of commercial polyol (i.e., the red curve).

### Example 6

The examples is directed to the bulk-scale, one-step synthesis of soybean polyols for use in rigid polyurethane foam production. Table 1 contains a description of the characteristics of various soybean polyols prepared by “click-ene” chemistry between 1-azidopropan-2-ol to the double bonds of soybean oil, selected for the preparation of rigid polyurethane foams (Scheme 8).

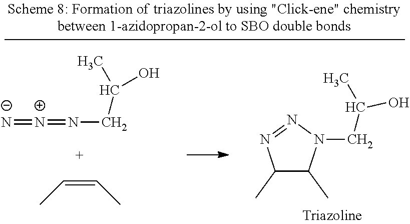

TABLE 1
Characteristics of polyols selected for
preparation of rigid polyurethane foams.

| | Hydroxyl | Acid | | | |
|---|---|---|---|---|---|
| | number | Value | | | |
| Sample | (mg KOH/g) | (mg KOH/g) | Mn | Mw | Mw/Mn |
| SB-OH | 193.63 | 3.82 | 284.20 | 760.32 | 2.67 |
| SB-OH-24 | 168.71 | 2.61 | 275.94 | 784.70 | 2.84 |
| Standard | 221.70 | 3.07 | 1253.05 | 2036.17 | 1.62 |
| Soy Polyol | | | | | |
| SB-OH-PO | 147.94 | 2.68 | 241.46 | 833.56 | 3.45 |

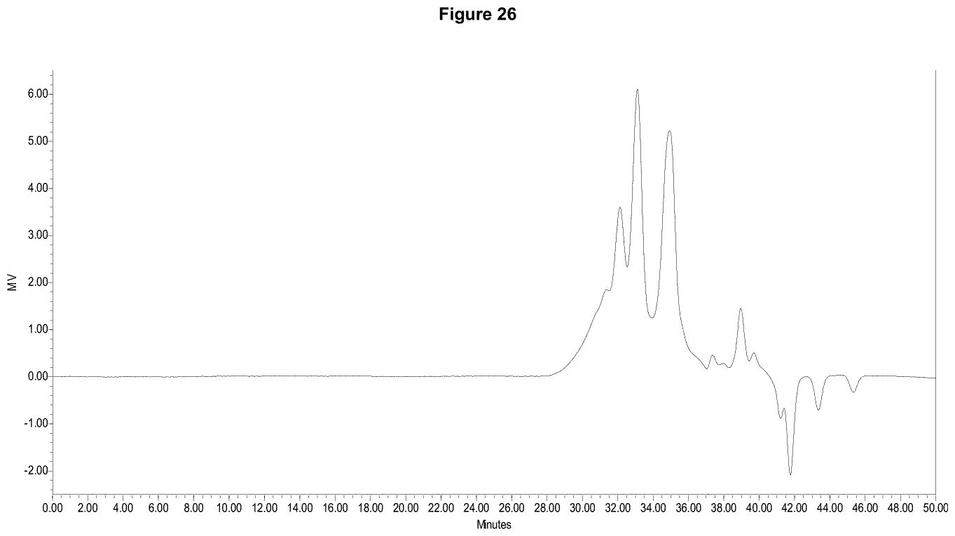

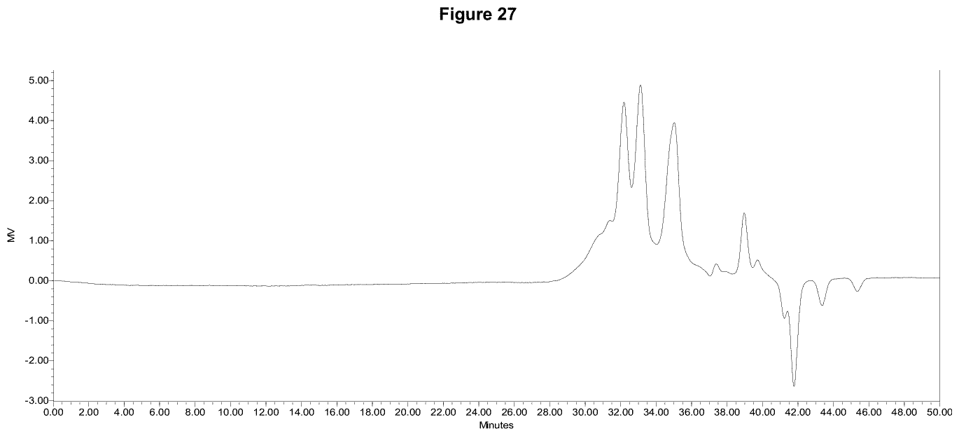

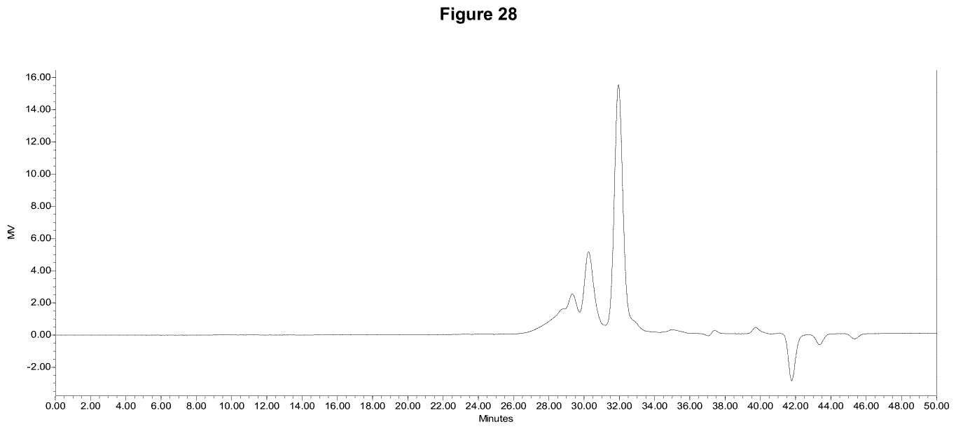

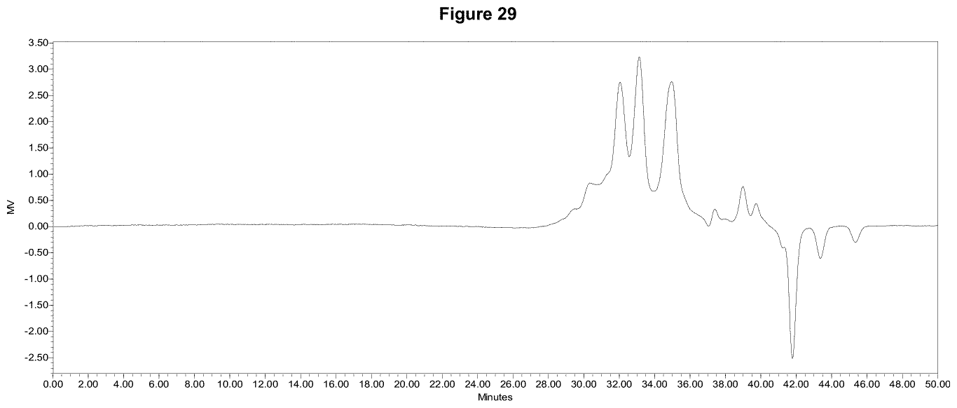

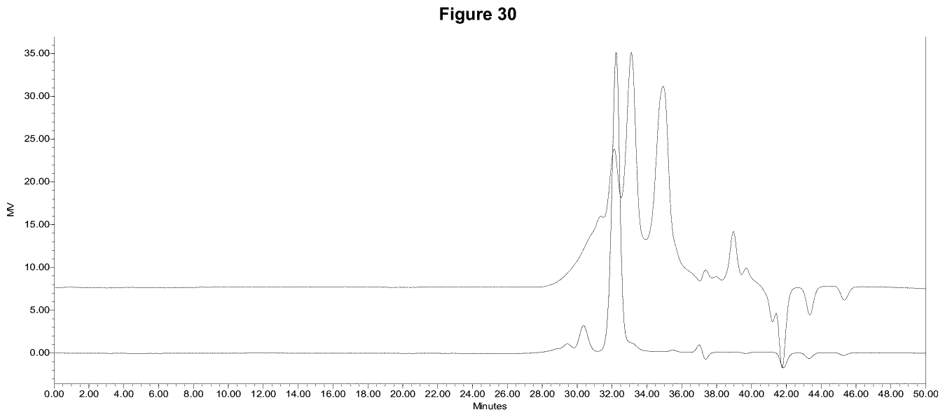

FIG. 26-30 show Gel Permeation Chromatograms of polyols from Table 1. FIG. 30 shows an overlay of chromatogram of soybean oil and of SBO polyol “SB-OH.” It is observed that wide-range of molecular species are present; probably monoglycerides, diglycerides, triglycerides, and polyols. As a result, it may be reasonably concluded that the reaction of this example produced a mixture of polyols. The exact composition of polyols may be established upon considering the 1H NMR, 13C NMR, and FT-IR spectra.

The synthesized click-ene polyols were used to prepare rigid polyurethane foams, which had the same or similar appearance as rigid polyurethane foams prepared using petrochemical polyols.

Preparation of rigid polyurethane foams: Normally the polyols used for preparation of rigid polyurethane foams have the hydroxyl number (OH#) in a range of 300-500 mg KOH/g. The polyols of Table 1 have an OH# in a range of 147-193 mg KOH/g. Due to this difference, the aza-“click” soybean polyol was mixed with another polyol having a higher hydroxyl number at a 50/50 weight ratio. Another set of foams was prepared by using as a soybean polyol produced by Cargill. Below is information regarding the compounds used prepare the rigid polyurethane foams:
Jeffol SG-520 is a polyether polyol based on sucrose with OH#=522 mg KOH/g;
Tegostab 8484 is a silicone surfactant for rigid PU foams;
DABCO BL-11 is a diamine ([(CH₃)₂NCH₂CH₂OCH₂CH₂N(CH₃)₂]) catalyst for reacting isocyanates with water;
DABCO-T-12 is a tin catalyst (dibutyl tin dilaurate) for reacting the hydroxyl groups of polyols with isocyanates;
Water is a chemical blowing agent generating gaseous carbon dioxide in the foaming process;
Rubinate 9257 is a polyisocyanate, a polymeric diphenyl diisocyanate of functionality of 2.9 isocyanate groups (—N═C═O)/mol.
   Seven rigid polyurethane foams were prepared: three from soybean aza “click” polyols mixed with Jeffol SG-520 (petrochemical polyether) and two foams based on soybean aza click polyols in mixture with the standard soybean polyol from Cargill.

The formulations used for these foams are presented below:

SANTRA-FOAM-1

| | | |
|---|---|---|
| Polyol SB-OH | 10.0 | g |
| Jeffol SG 520 | 10.0 | g |
| Silicon B-8484 | 0.4 | g |
| DABCO BL-11 | 0.12 | g |
| DABCO T-12 | 0.04 | g |
| Water | 0.8 | g |
| Total formulated polyol(A) | 21.36 | g |
| Rubinate 9257 (index 105) (B) | 29.7 | g |
| B/A = 1.39 | | |

SANTRA-FOAM-2

| | | |
|---|---|---|
| Polyol SB-OH-PO | 10.0 | g |
| Jeffol SG 520 | 10.0 | g |
| Silicon B-8484 | 0.4 | g |
| DABCO BL-11 | 0.12 | g |
| DABCO T-12 | 0.04 | g |
| Water | 0.8 | g |
| Total formulated polyol(A) | 21.36 | g |
| Rubinate 9257 (index 105) (B) | 28.6 | g |
| B/A = 1.33 | | |

SANTRA-FOAM -3

| | | |
|---|---|---|
| Standard polyol | 10.0 | g |
| Jeffol SG 520 | 10.0 | g |
| Silicon B-8484 | 0.4 | g |
| DABCO BL-11 | 0.12 | g |
| DABCO T-12 | 0.04 | g |
| Water | 0.8 | g |
| Total formulated polyol(A) | 21.36 | g |
| Rubinate 9257 (index 105) (B) | 28.6 | g |
| B/A = 1.33 | | |

SANTRA-FOAM-4

| | | |
|---|---|---|
| Polyol SB-OH | 10.0 | g |
| Standard polyol | 10.0 | g |
| Silicon B-8484 | 0.4 | g |
| DABCO BL-11 | 0.12 | g |
| DABCO T-12 | 0.04 | g |
| Water | 0.8 | g |
| Total formulated polyol(A) | 21.36 | g |
| Rubinate 9257 (index 105) (B) | 23.1 | g |
| B/A = 1.08 | | |

SANTRA-FOAM-5

| | | |
|---|---|---|
| Polyol SB-OH-PO | 10.0 | g |
| Standard polyol | 10.0 | g |
| Silicon B-8484 | 0.4 | g |
| DABCO BL-11 | 0.12 | g |
| DABCO T-12 | 0.04 | g |
| Water | 0.8 | g |
| Total formulated polyol (A) | 21.36 | g |
| Rubinate 9257 (index 105) (B) | 22.0 | g |
| B/A = 1.03 | | |

SANTRA-FOAM-6

| | | |
|---|---|---|
| SB-Polyol-24 h | 10 | g |
| Jeffol SG 520 | 10 | g |
| Tegostab 8484 | 0.4 | g |
| DABCO BL 11 | 0.12 | g |
| DABCO T-12 | 0.04 | g |
| Water | 0.8 | g |
| Total formulated polyol(A) | 21.36 | g |
| Rubinate 9257 (index 105) (B) | 29.27 | g |
| B/A = 1.37 | | |

SANTRA-FOAM-7

| | | |
|---|---|---|
| SB-Polyol-24 h | 10 | g |
| Std. Polyol | 10 | g |
| Silicon 8484 | 0.4 | g |
| Niax A-1 | 0.12 | g |
| T-12 | 0.04 | g |
| Water | 0.8 | g |
| Total formulated polylol(A) | 21.36 | g |
| Rubinate 9257 (index 105) (B) | 20.95 | g |
| B/A = 0.98 | | |

The polyols, silicon surfactant, catalysts, and water were mixed to obtain the polyol component A. The component A was mixed vigorously with a stirrer at 3500 revolutions/min with the isocyanate Rubinate 9257. The cream time, rise time, and tack-free time were recorded.

Cream time is the moment during the mixing at which the formulated polyol and the isocyanate starts to foam. Rise time is the moment during the mixing at which the foam rises to a maximum height (i.e., the rising stops). Tack-free time is the moment at which the foam become not tacky. Cream time, rise time and tack-free time recorded during preparation of mentioned five foams are presented in Table 2 and Table 3 below. Without being bound to a particular theory, it is believed that the rise times are relatively short due to a possible catalytic effect of the azide group.

TABLE 2
Foaming times for aza-click soy polyols
in mixture with polyol Jeffol SG-520

| Name | Cream Time | Rise Time | Tack-free Time |
|---|---|---|---|
| Foam-1 | 8 | 43 | 58 |
| Foam-2 | 8 | 24 | 24 |
| Foam-3 | 7 | 38 | 52 |

TABLE 3
Foaming times for aza click soy polyols
in mixture with standard soy polyol.

| Name | Cream Time | Rise Time | Tack-free Time |
|---|---|---|---|
| Foam -4 | 7 | 30 | 54 |
| Foam-5 | 8 | 42 | 51 |
| Foam-6 | 7 | 30 | 54 |
| Foam-7 | 8 | 42 | 51 |

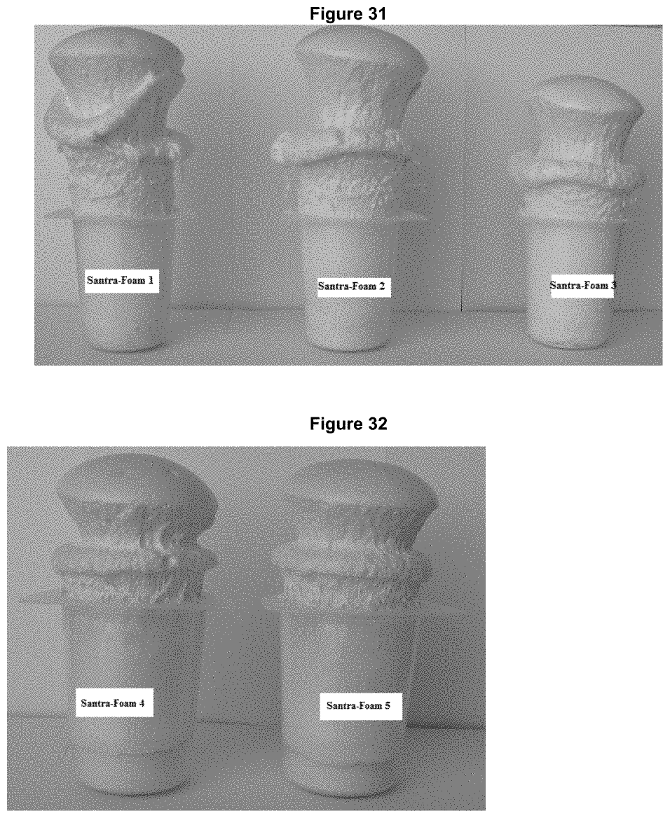

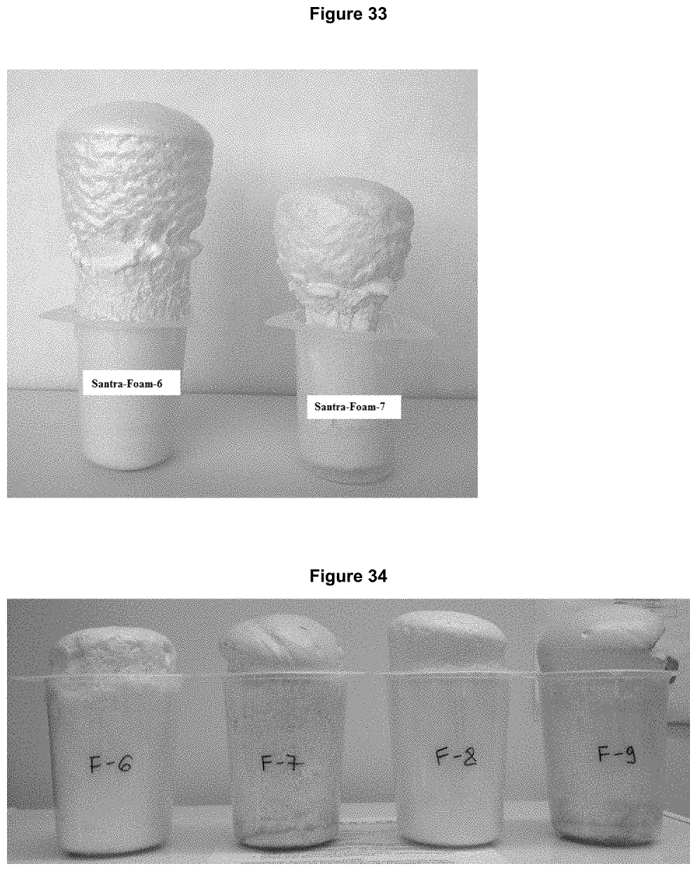

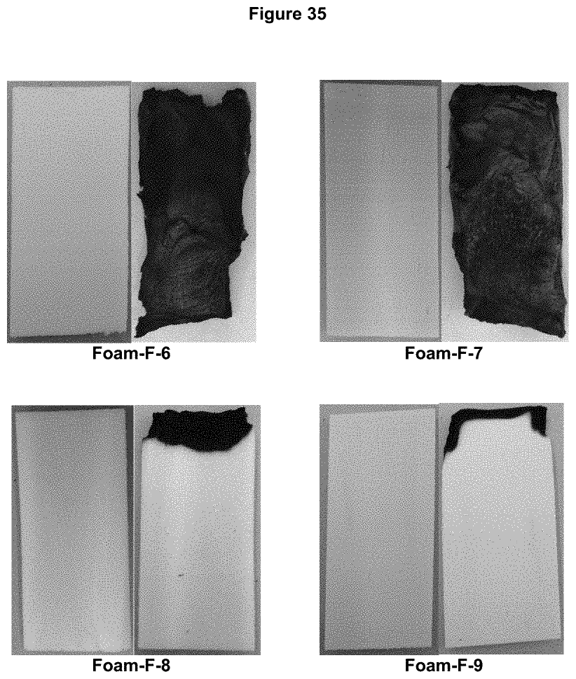

The images of rigid polyurethane foams prepared with aza “click” soybean polyols are presented in FIGS. 31-33.

The physical/mechanical properties of resulting rigid polyurethane foams produced from soybean polyols were similar to that produced from petrochemical polyols, as it is observed in Table 4.

TABLE 4
Characteristics of rigid polyurethane foams based on
synthesized aza-“click” soy polyols.

| | Density^{a} | Density^{b} | | Closed | Compression | |
|---|---|---|---|---|---|---|
| | Cube | Cylinder | Average | cell | strength @ | |
| Foam | shape | shape | Density | content | 10% strain | Tg |
| ID | (kg/m³) | (kg/m³) | (kg/m³) | (%) | (kPa) | (° C.) |
| Foam 1 | 33 | 35 | 34 | 90 | 191 | 81.08 |
| Foam 2 | 31 | 32 | 31.5 | 94 | 144 | 66.42 |
| Foam 3 | 42 | 41 | 41.5 | 91 | 239 | 86.62 |
| Foam 4 | 51 | 49 | 50 | 92 | 219 | 61.18 |
| Foam 5 | 48 | — | — | — | 193 | 51.85 |
| Foam 6 | 30 | 30 | 30 | 49 | 155 | 28.64 |
| Foam 7 | 32.5 | — | — | — | 105 | 39.34 |

The synthesized aza-“click” soy polyols mixed 50/50 with a second polyol of a higher hydroxyl number produced rigid polyurethane foams with desirable properties.

Effect of temperature on one-step click-ene SBO polyol synthesis: Polyol synthesis using the click-ene reaction was conducted at 70° C. and 120° C. (“Low T” and “High T,” respectively). These two soybean polyols were used for the formulation of rigid PU foams using the following protocols:

TABLE 5
Formulations for rigid PU foams using these two polyols
prepared by “click-ene” chemistry.

| | OH | |
|---|---|---|
| Polyol | (mg KOH/g) | Equivalent weight |
| SBO-Polyol Low T | 167.43 | 335.12 |
| SBO Polyol-48 h-High T | 214.32 | 261.80 |
| Jeffol-SG-520 | 520.00 | 107.90 |
| Isocyanate | | |
| Rubinate M | — | 135.00 |

Formulation F-6

| | | |
|---|---|---|
| SBO-Polyol Low T | 10.0 | g |
| Jeffol SG-520 | 10.0 | g |
| Tegostab 8484 | 0.4 | g |
| DABCO BL-11 | 0.12 | g |
| DABCO T-12 | 0.04 | g |
| Water | 0.80 | g |
| Total A | 21.35 | g |
| Rubinate M (index 105) | 29.5 | g |

Formulation F-7

| | | |
|---|---|---|
| SBO-Polyol 48 h-High T | 10.0 | g |
| Jeffol SG-520 | 10.0 | g |
| Tegostab 8484 | 0.4 | g |
| DABCO BL-11 | 0.12 | g |
| DABCO T-12 | 0.04 | g |
| Water | 0.80 | g |
| Total A | 21.35 | g |
| Rubinate M (index 105) | 30.7 | g |

Formulation F-8

| | | |
|---|---|---|
| SBO-Polyol Low T | 10.0 | g |
| Jeffol SG-520 | 10.0 | g |
| Tegostab 8484 | 0.4 | g |
| TCEP | 8.6 | g |
| DABCO BL-11 | 0.12 | g |
| DABCO T-12 | 0.04 | g |
| Water | 0.80 | g |
| Total A | 29.95 | g |
| Rubinate M (index 105) | 29.5 | g |

Formulation F-9

| | | |
|---|---|---|
| SBO-Polyol 48 h-High T | 10.0 | g |
| Jeffol SG-520 | 10.0 | g |
| Tegostab 8484 | 0.4 | g |
| TCEP | 8.6 | g |
| DABCO BL-11 | 0.12 | g |
| DABCO T-12 | 0.04 | g |
| Water | 0.80 | g |
| Total A | 29.95 | g |
| Rubinate M (index 105) | 30.7 | g |

Rigid polyurethane foams were prepared as follows: Initially, a mixture of polyols, siliconic surfactant, catalysts, and water was prepared. The mixture is called Component A. To component A was added the isocyanate (Rubinate M) and the mixture was stirred at 3000 revolutions/min. The cream time occurred at about 10 seconds. The rise times occurred in a range of about 20 to 30 seconds. The foams were stored at room temperature around one week, and during this time the unreacted isocyanate groups react in the solid foams. After being so stored, the following properties were determined: density, closed cell content and compression strength at 10% deformation.

FIG. 34 contains images of the foams prepared with formulations F-6, F-7, F-8 and F-9.

TABLE 6
Characterization of foams F-6 to F-9.

| | | Closed cell | Compression strength at |
|---|---|---|---|
| | Density | content | 10% deformation |
| Sample | (kg/m³) | (%) | (kPa) |
| PU foam-F-6 | 39.4 | 24 | 82.9 |
| PU foam-F-7 | 37.4 | 93 | 170.1 |
| PU foam-F-8 | 46.9 | 12 | 151.8 |
| PU foam-F-9 | 44.4 | 91 | 214.3 |

The foams F-6 and F-8 were prepared with the polyol synthesized at 70° C. (SBO Polyol Low T), and the foams F-7 and F-9 with the polyol prepared at higher temperature (120° C.) for 48 hours (SBO-Polyol 48 h High T). It is observed that the foams based on the polyol synthesized at lower temperature had less desirable properties—a relatively low closed cell content of 12% and 24% whereas typical thermos/insulation foams >90%. Additionally, foam F-6 had a relatively low compression strength of 82 kPa whereas typical foams have a minimum compression strength of 120 kPa. In contrast, the polyol synthesized at higher temperature yielded foams with a relatively high closed cell content of about 91-93%, and a relatively high compression strength of 170 kPa for foam F-7 and 214 kPa for foam F-9.

### Example 7: Synthesis of Flame Retarded Foams

Flame retardant foams were prepared with low- and high-temperature polyols. These foams contained tris (2-chloroethyl) phosphate (TCEP) as a flame retardant with Foams 8 and 9 also containing about 10.8% of phosphorus. Foams 8 and 9 qualify as flame retardant foams because the their self-extinguishing times (or burning times) were less than 1 minute (i.e., 55 seconds for foam F-8 and 32 seconds for foam F-9).

The flammability characteristics of foams F-6, F-7, F-8 and F-9 are presented in Table 7. Foams without flame retardant (TCEP) burned completely.

TABLE 7
Flammability characteristics of PU foams F-6, F-7, F-8 and F-9

| Sample | WBB (g) | WAB (g) | WLOSS (%) | BT (s) |
|---|---|---|---|---|
| SANTRA-F-6 | 3.174 | 1.260 | 60.3 | 88 |
| SANTRA-F-7 | 3.475 | 2.105 | 39.4 | 105 |
| SANTRA-F-8 | 4.002 | 3.548 | 11.3 | 55 |
| SANTRA-F-9 | 4.304 | 3.957 | 8.0 | 32 |

WBB = weight before burning; WAB = weight after burning; WLOS = weight lost after burning; BT = burning time.

The foams made using the polyol prepared at higher temperature and long reaction time (SBO Polyol 48H-High T) performed better than those made using the polyol prepared at low temperature (SBO-Polyol Low T). Specifically, the high T polyol foams had better physical/mechanical characteristics and superior flame retardant properties.

### Example 8: Cast Polyurethanes from Soybean Polyols Prepared by “Aza Click” Reactions

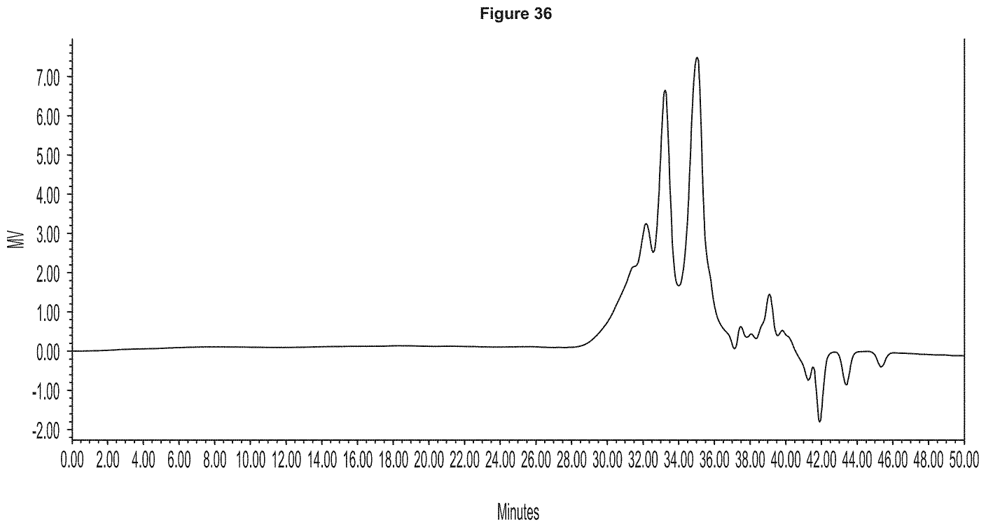

Cast polyurethanes were produced by the direct reaction of polyols with polyisocyanates in the absence of any catalysts or blowing agents. The homogeneous mixture polyisocyanate with the polyol was poured in a mold and it was heated several hours at 110° C. in an oven. After this period of heating, the mixture had become a rigid polyurethane. Rubinate 9257 was the polyisocyanate that was used. It is an isocyanate with functionality of 2.9 —N═C═O groups/mol. The isocyanate index of 105 was used. SB-OH-PO-5 (the fifth sample of aza polyol) was used as the polyol. It was made using soybean oil and 2-hydroxypropyl azide (HPA), which were reacted for 48 hours at 120° C. The characteristics of the polyol used for cast PU are presented in Table 8 and the GPC chromatogram of polyol in FIG. 36. In GPC chromatogram are observed molecular species of lower molecular weight than triglycerides, probably diglycerides and monoglycerides.

TABLE 8
Characteristics of aza polyol SB-OH-PO-5 used
for preparation of cast polyurethanes

| | Viscosity | OH # | Acid Value | | | |
|---|---|---|---|---|---|---|
| Sample | (Pa · s) | (mg KOH/g) | (mg KOH/g) | Mn | Mw | Mw/Mn |
| SB-OH-PO-5 | 0.515 | 128.63 | 2.78 | 323.31 | 762.70 | 2.36 |
| (48 h reaction at | | | | | | |
| 120° C.) | | | | | | |

The formulations used for preparation of three cast PU are presented below:

### Cast 1.

| | | | |
|---|---|---|---|
| 1. | SB-OH-PO-5 polyol (OH# = | 18.9 | g |
| | 128 mg KOH/g): | | |
| 2. | Rubinate 9257: | 6.4 | g (index 105) |
| | | 25.3 | g |

### Cast 2.

| | | | |
|---|---|---|---|
| 1. | SB-OH-PO-5 polyol (OH# = | 8.7 | g |
| | 128 mg KOH/g): | | |
| 2. | Jeffol SG 520: | 8.7 | g |
| 3. | Rubinate 9257: | 7.9 | g (index 105) |
| | | 25.3 | g |

### Cast 3.

| | | | |
|---|---|---|---|
| 1. | SB-OH-PO-5 polyol (OH# = | 12.8 | g |
| | 128 mg KOH/g): | | |
| 2. | Glycerol: | 1.43 | g |
| 3. | Rubinate 9257: | 11.23 | g (index 105) |
| | | 25.46 | g |

For simplification of terminology, the azide-based polyol is referred to as a click-ene polyol. Cast 1 was prepared by using only the SB-OH-PO-5 aza polyol and polyisocyanate Rubinate 9257. Cast 2 was prepared using a mixture between aza polyol SB-OH-PO-5 and sucrose polyol Jeffol-SG-520 to improve the crosslink density and the same isocyanate Rubinate 9257. Cast 3 was prepared by using the aza polyol SB-OH-PO-5 together with glycerol as crosslinker and the isocyanate 9257.

Each of the cast PUs demonstrated a cellular structure. It is believed that the cellular structure is due to gaseous nitrogen generated by the decomposition of triazinic ring remaining after the synthesis.

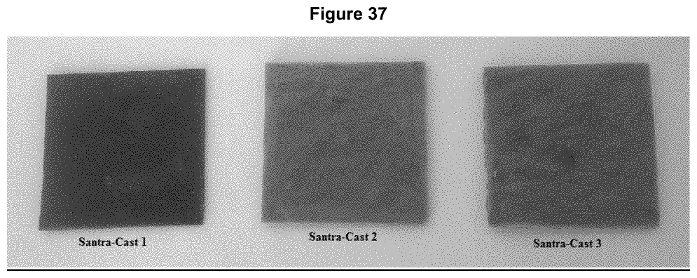

FIG. 37 contains photos of these three cast polyurethanes obtained with the aza polyol SB-OH-PO-3. The color is due to the dark color of polyol maintained 48 hours at 120° C. As is discussed below, the dark color is substantially improved if a lower temperature and oxygen atmosphere are used to make the polyol.

FIG. 37 also contains images of the cast PU prepared with aza polyol SB-OH-PO-5. Table 9 sets forth some characteristics if these cast polyurethanes.

TABLE 9
Characteristics of cast PU prepared with aza polyol SB-OH-PO-5.

| | | | Tensile | Break | Tangent |
|---|---|---|---|---|---|
| | Tg | Hardness | Strength | Elongation | Modulus |
| Sample | (° C.) | (Shore A) | (MPa) | (%) | (MPa) |
| Cast 1 | 27.44 | 30 | 94.1 | 0.230 | 1.059 |
| Cast 2 | 15.19 | 65 | — | — | 15.99 |
| Cast 3 | 42.82 | 70 | — | — | — |

Some characteristics of Cast 2 and Cast 3 were not possible to be determined due to the high thickness generated by cellular structure.

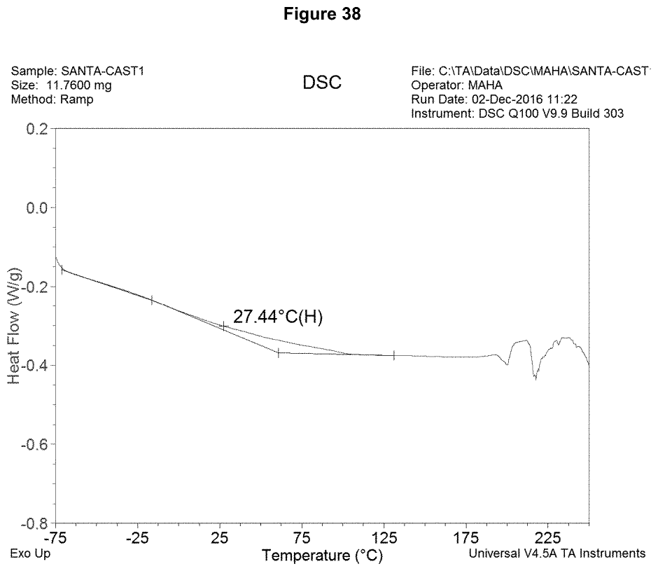

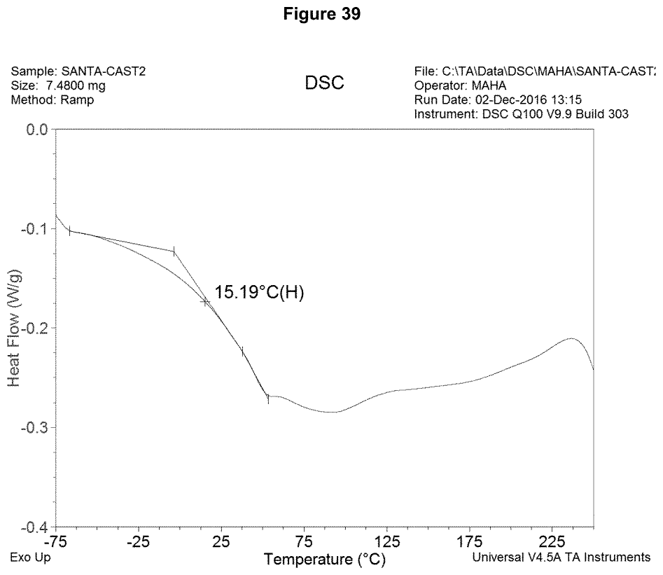

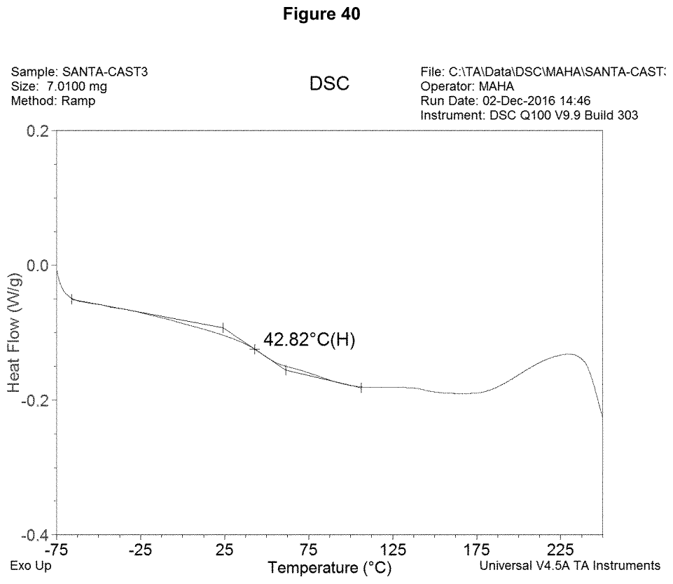

FIGS. 38-40 contain Differential calorimetry (DSC) curves of Cast 1, Cast 2 and Cast 3, in which the Glass Transition Temperatures (Tg) are displayed.

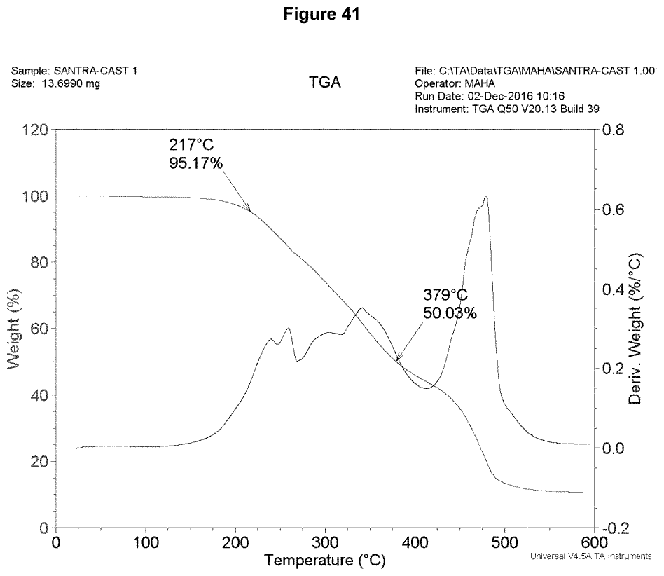

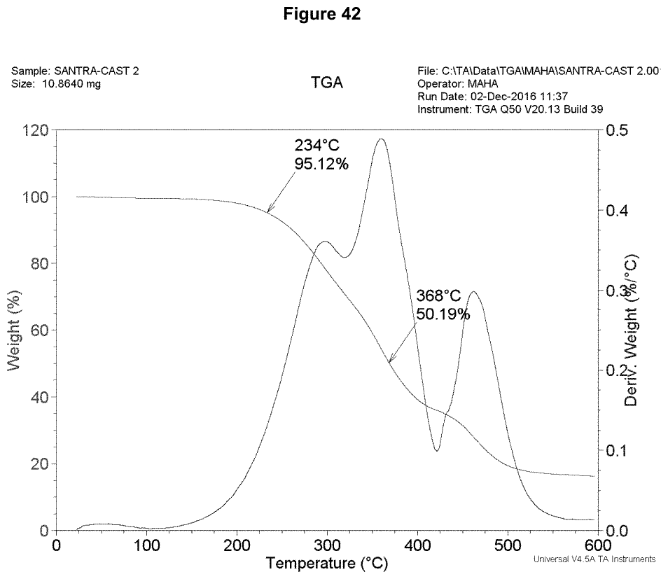

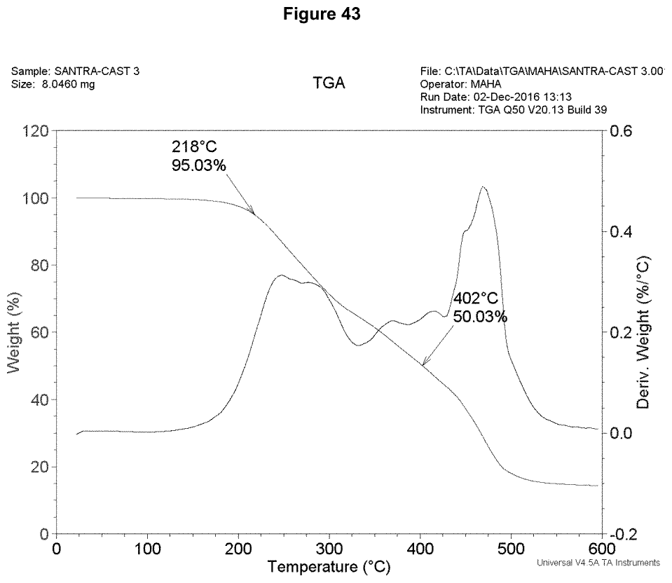

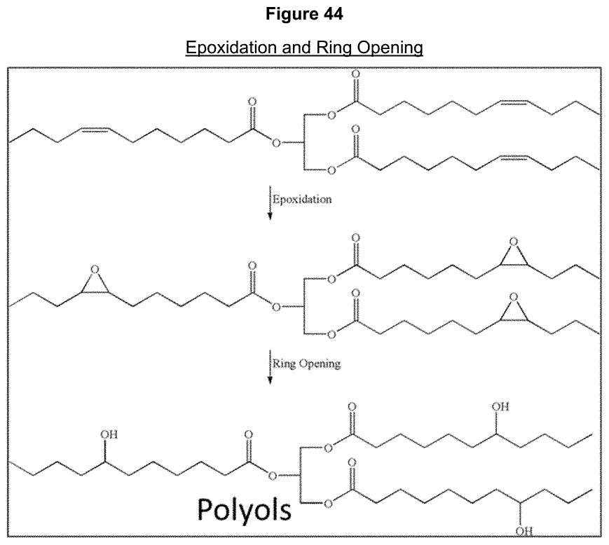

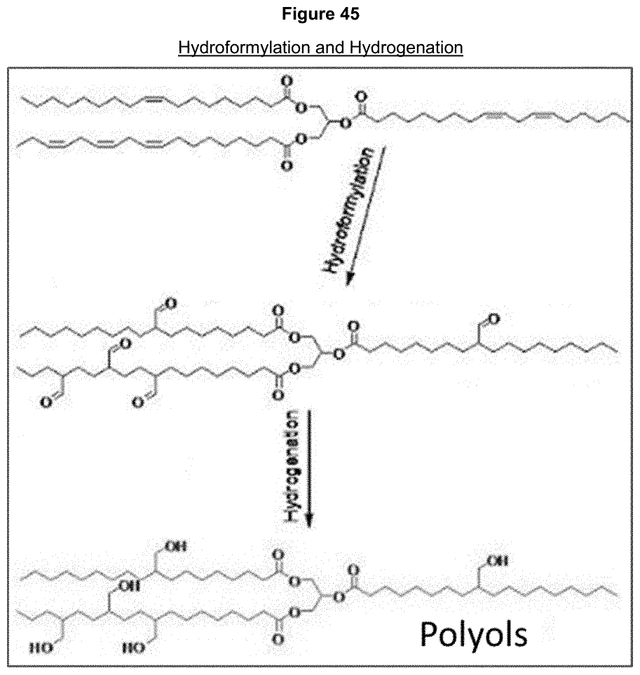

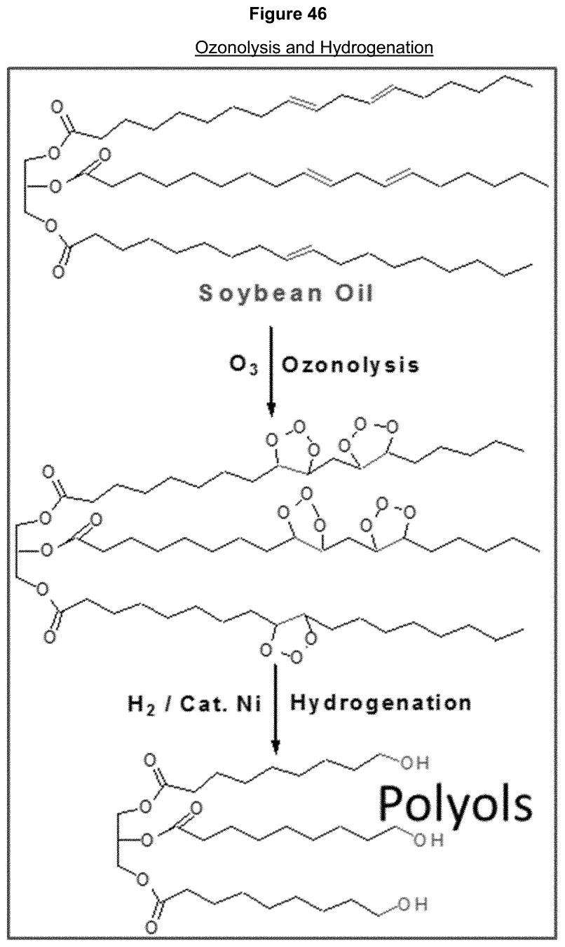

FIGS. 41-43 present the thermogravimetric (TGA) analyses of Cast 1, Cast 2 and Cast 3. All three cast PUs decomposed at temperatures higher than 200° C. (between 217-234° C.), which indicates that the polyurethanes based on polyol SB-OH-PO-5 had good thermal stabilities. The hardness of Cast 2 and Cast 3 is greater than Cast 1 due to utilization of cross-linkers.

### Example 9

This example is a one-step synthesis of soybean polyols from propylene oxide azide and soybean oil using the “Click-ene” chemistry and UV light without using any solvent or catalyst according to Scheme 9.

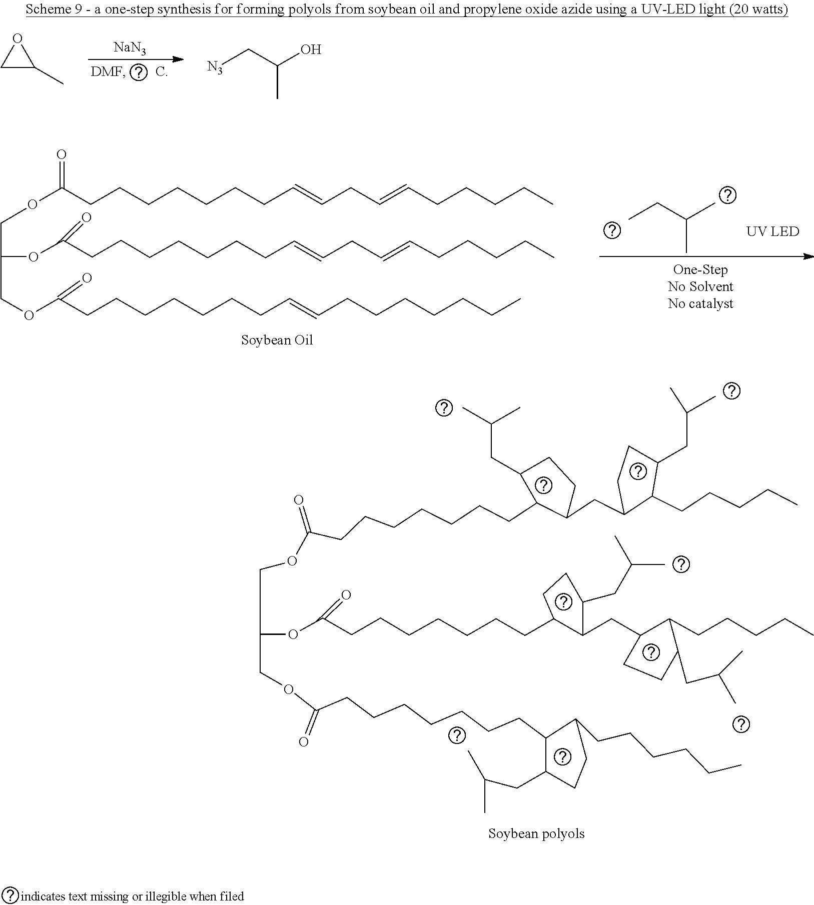

Procedure: The reaction between propylene oxide azide and soybean oil using a 20 watt UV-LED without using any solvent or catalyst. Briefly, propylene oxide azide (4.8 mol) and soybean oil (1 mol) were added to a 50-mL round-bottom flask (Scheme 9). The reaction mixture was subjected to UV light with stirring. Upon completion of the 72 hours, the reaction was stopped. Samples were collected at different time intervals and characterized using various spectroscopic methods to determine the progress of the reaction.

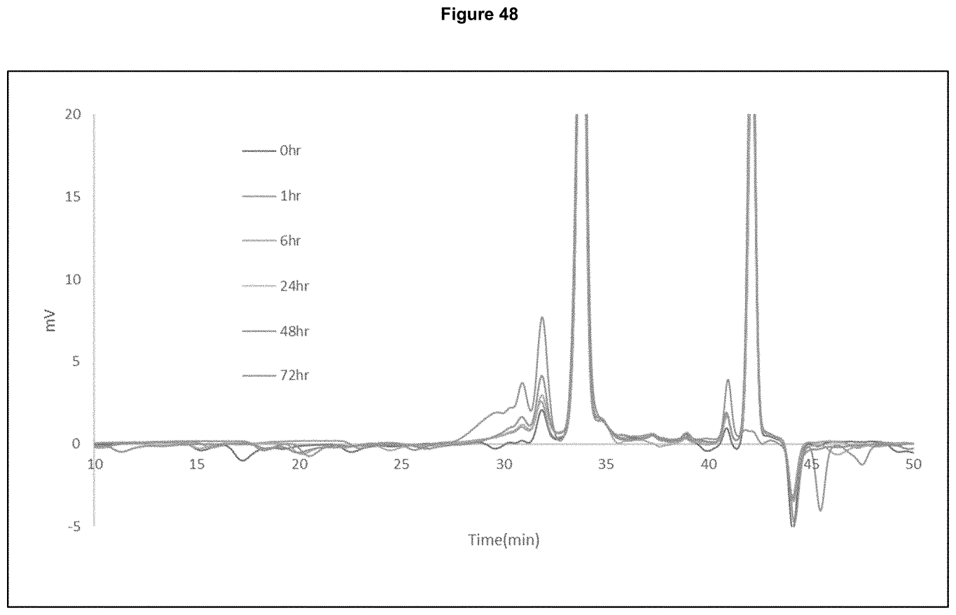

The synthesized soybean polyols at different time points were characterized by gel permeation chromatography, as shown in FIG. 48. The appearance of new band over time shows the formation of a new product (i.e., the expected soybean polyols). After 72 hours of reaction, no substantial change in the GPC chromatograms were observed, which indicates that the reaction was completed within 72 hours.

### Example 10

This example is a one-step synthesis of soybean polyols from propylene oxide azide and soybean oil using the “Click-ene” chemistry and microwave without using any solvent or catalyst according to Scheme 10.

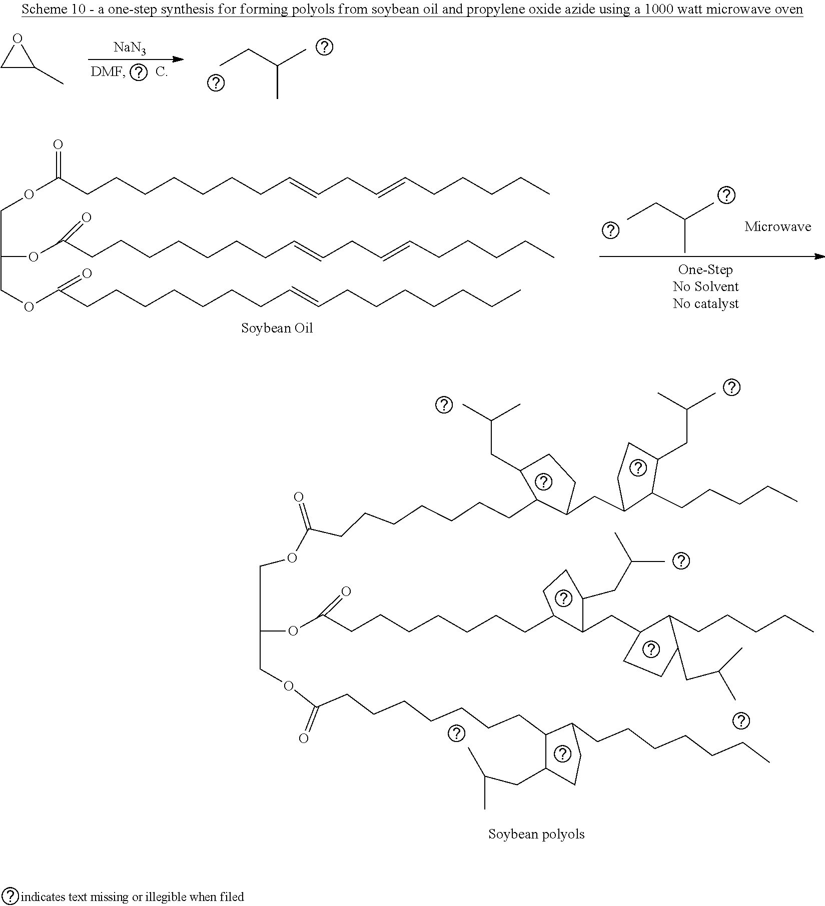

Procedure: The reaction between propylene oxide azide and soybean oil using a 1000 watt microwave oven without using any solvent or catalyst. Briefly, propylene oxide azide (4.8 mol) and soybean oil (1 mol) were added to a 50-mL round-bottom flask (Scheme 10). The reaction mixture was subjected to microwaves with stirring. Upon completion of the 15 minutes, the reaction was stopped. Samples were collected at different time intervals and characterized using various spectroscopic methods to determine the progress of the reaction.

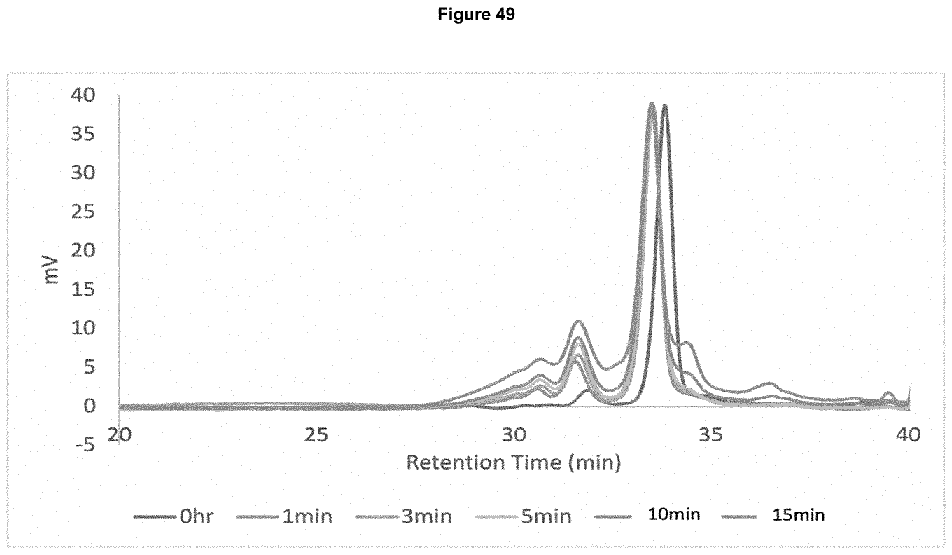

The synthesized soybean polyols at different time points were characterized by gel permeation chromatography, as shown in FIG. 49. The appearance of new band over time shows the formation of a new product (i.e., the expected soybean polyols). After 15 minutes of reaction, no substantial change in the GPC chromatograms were observed, which indicates that the reaction was completed within 15 minutes.

Having illustrated and described the principles of the present invention, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of producing a triazoline-containing compound, the method comprising reacting an alkene, which comprises at least one a C═C double bond, with an azido compound, which comprises an azide anion having the chemical formula N₃⁻, wherein the alkene and the azido compound are constituents of a reaction mixture, so that a C—C single bond forms between the carbon atoms of the at least one C═C double bond and each carbon atom of the C—C single bond also has a single bond with a different nitrogen atom of the azide anion thereby producing the triazoline-containing compound according to Scheme I, 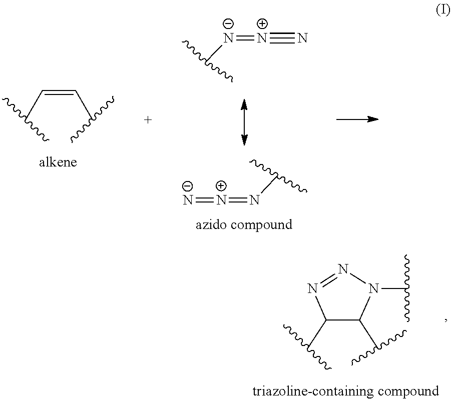
wherein the triazoline-containing compound is a constituent of a product mixture.

2. The method of claim 1, wherein the alkene is selected from the group consisting of triglycerides, small molecule aliphatic alkenes, terminal alkenes, and combinations thereof.

3. The method of claim 2, wherein the triglycerides are one or more vegetable oils selected from the group consisting of soybean oil, corn oil, palm oil, sunflower oil, canola oil, sesame oil, peanut oil, olive oil, cottonseed oil, avocado oil, almond oil, walnut oil, flaxseed oil, and combinations thereof.

4. The method of claim 1, wherein the azido compound further comprises a functional group selected from the group consisting of a hydroxyl group, an alkyl group, an amine group, a thiol group, and an ether group.

5. The method of claim 1, wherein azido compound further comprises a hydroxyl functional group and is selected from the group consisting of propylene oxide azide, alkyl azides, alkane diazides, functional alkyl azides, and combinations thereof.

6. The method of claim 1, wherein the reaction mixture is free of a solvent.

7. The method of claim 1, wherein the reaction mixture is free of any other reagent.

8. The method of claim 1, wherein the reaction mixture is free of a catalyst.

9. The method of claim 1, wherein the reaction mixture is free of an initiator.

10. The method of claim 1, wherein the reaction mixture consists of the alkene and the azido compound.

11. The method of claim 1, wherein the step of reacting the alkene and the azido compound comprises adding an effective amount of energy to the reaction mixture to cause the reaction between the alkene and the azido compound for a desired duration.

12. The method of claim 11, wherein the effective amount of energy added to the reaction mixture to cause the reaction between the alkene and the azido compound is achieved by maintaining the reaction mixture at a temperature in a range of about 75° C. to about 180° C. and the desired duration is in a range from about 12 hours to about 48 hours.

13. The method of claim 11, wherein the effective amount of energy added to the reaction mixture to cause the reaction between the alkene and the azido compound is achieved by exposing the reaction mixture to ultraviolet light with a wavelength in a range of about 200 nm to about 400 nm at a power in a range of about 20 watts to about 200 watts per 10 grams to 1,000 grams of reaction mixture and the desired duration is in a range from about 12 to about 72 hours.

14. The method of claim 11, wherein the effective amount of energy added to the reaction mixture to cause the reaction between the alkene and the azido compound is achieved by exposing the reaction mixture to microwave irradiation with a wavelength in a range about 1×10⁶ nm to about 1×10⁸ nm at a power in a range of about 700 wats to about 1,200 watts per 1 gram to 100 grams of reaction mixture for a duration in a range of about 5 minutes to about 30 minutes.

15. The method of claim 1, wherein the product mixture contains at least 90% by weight of the triazoline-containing compound.

16. A triazoline-containing triglyceride molecule comprising a glycerol-based backbone moiety and three fatty acid-based chain moieties bound to the glycerol-based backbone moiety via ester bonds, wherein at least one of the fatty acid-based chain moieties comprises at least one triazoline moiety that comprises a 5-membered heterocycle ring of two carbon atoms and three nitrogen atoms, wherein the two carbon atoms are also adjacent carbon atoms of the fatty acid-based chain moiety.

17. The triazoline-containing triglyceride molecule of claim 16, wherein the at least one triazoline moiety further comprises a functional group selected from the group consisting of a hydroxyl group, an alkyl group, an amine group, a thiol group, and an ether group.

18. The triazoline-containing triglyceride molecule of claim 16, wherein the at least one triazoline moiety comprises a functional group that is a hydroxyl group.

19. The triazoline-containing triglyceride molecule of claim 16, wherein two of the three fatty acid-based chain moieties comprise at least one triazoline moiety.

20. The triazoline-containing triglyceride molecule of one claim 16, wherein each of the three fatty acid-based chain moieties comprises at least one triazoline moiety.

21. The triazoline-containing triglyceride molecule of claim 16, wherein the at least one triazaoline moiety further comprises a linking moiety between the 5-membered heterocycle ring and the functional group, wherein the linking moiety is selected from the group consisting of alkyl and aryl azide derivatives.

22. A vegetable oil-based polyol molecule comprising:
a triglyceride moiety that comprises a glycerol-based backbone and three fatty acid-based chains bound to the glycerol-based backbone via ester bonds; and
at least one triazoline moiety that comprises:
a 5-membered heterocycle ring of two carbon atoms and three nitrogen atoms in which the two carbon atoms are also adjacent carbon atoms of one of the fatty acid-based chains of the triglyceride moiety; and
a hydroxyl functional group.

23. The vegetable oil-based polyol molecule of claim 22, wherein the vegetable oil is selected from the group consisting of soybean oil, corn oil, palm oil, sunflower oil, canola oil, sesame oil, peanut oil, olive oil, cottonseed oil, avocado oil, almond oil, walnut oil, flaxseed oil, and combinations thereof.

24. The vegetable oil-based polyol molecule of claim 22, wherein the vegetable oil is soybean oil.

25. The vegetable oil-based polyol molecule of claim 22 comprising at least one triazoline moiety with each fatty acid-based chain of the triglyceride portion.

26. The vegetable oil-based polyol molecule of claim 22, wherein the at least one triazaoline moiety further comprises a linking moiety between the 5-membered heterocycle ring and the functional group, wherein the linking moiety is selected from the group consisting of alkyl and aryl azide derivatives.
